(19) United States
(12) Patent Application Publication
(10) Pub. No.: US 2008/0317761 A1
(43) Pub. Date: Dec. 25, 2008

(54) Peptide-Mediated Protein Transduction Into Cells of the Hematopoietic Lineage

(75) Inventors: Douglas B. Cines, Wynnewood, PA (US); Mortimer Poncz, Wynnewood, PA (US)

Correspondence Address: HOWSON AND HOWSON, SUITE 210, 501 OFFICE CENTER DRIVE, FT WASHINGTON, PA, 19034, US

(73) Assignees: The Trustees of the University of Pennsylvania, Philadelphia, PA (US); The Children's Hospital of Philadelphia, Philadelphia, PA (US)

(21) Appl. No.: 11/587,873

(22) PCT Filed: Apr. 28, 2005
(86) PCT No.: PCT/US2005/014445

§ 371 Date: Oct. 27, 2006

Related U.S. Application Data
(60) Provisional application No. 60/565,940, filed on Apr. 28, 2004.

(51) Int. Cl. A61K 39/395; A61K 35/12; A61P 9/00

(52) U.S. Cl. 424/159.1; 424/93.7; 424/93.71; 424/93.72

(57) ABSTRACT

A pharmaceutical composition comprises a therapeutic peptide or protein, a transport moiety capable of transporting said first peptide or protein into a hematopoietic cell differentiated from a common myeloid progenitor, and a linker between said first protein and said transport moiety, said linker susceptible to cleavage by an intracellular enzyme in the cell. A cell or collection of cells, e.g., platelets, containing such a composition is useful in methods for treating infection, inflammation, vascular injuries or any disorders involving or mediated by cells of the hematopoietic lineage. Methods of making such compostions are also disclosed.

### STATEMENT REGARDING FEDERALLY SPONSORED RESEARCH OR DEVELOPMENT

The present invention was supported, in part, by the National Institutes of Health, Grant Nos. PO1 HL64190, DBR RO1 HL60169, and K08 HL67913-01. The United States government has an interest in this invention.

### SEQUENCE LISTING

The sequence listing appearing at the end of this application contains reference to a specific amino acid linker employed in the examples as SEQ ID NO: 1 and a nucleotide sequence for the exemplary construct as SEQ ID NO: 2.

### BACKGROUND OF THE INVENTION

A number of cells of hematopoietic lineage are secretory cells upon activation. These cells can participate in both the advance of disease or the prevention thereof based on the molecules they secrete. For example, platelets, the smallest corpuscular components of human blood, are characterized by a diameter of about 2-4 micrometers, the absence of a nucleus, and a physiological number varying from 150,000 to 300,000 per cubic millimeter of blood. Platelets contribute to the complex, multistep, and highly regulated process of thrombus formation and arterial occlusive disorders, a leading cause of human morbidity. Platelets target and adhere to sites of vascular injury. At the sites of vascular injury, the platelets are activated and form aggregates that provide a provisional seal.

Platelets preferentially release their granular contents at the site of injury, e.g., contributing to the subsequent growth and stability of thrombi in part through the release of von Willebrand factor (vWF), fibrinogen, and other coagulation proteins such as Factor V (Holt J. C., and Niewiarowski, S. 1985 Sem. Hematol. 22:151-163) from their alpha-granules. Activated platelets also release proteins that inhibit thrombolysis, chief among which is plasminogen activator inhibitor-1 (PAI-1). Over 90% of the circulating PAI-1 is stored in platelet alpha-granules (Booth, N. A et al, 1988 Brit. J. Haematol. 70:327-333). Much of the PAI-1 is in an inactive form (Declerck, P. J et al, 1988 Blood 71:220-225; Kruithof, E. K et al, 1987 Blood 70:1645-1653). Nonetheless, this pool of PAI-1 is thought to be one of the main reasons why platelet-rich thrombi are especially resistant to thrombolytic therapy (Booth, N. A et al, 1992 Ann. N.Y. Acad. Sci. 667:70-80; Fay, W. P et al, 1994 Blood 83:351-356).

Paradoxically, platelets also contain or can bind small amounts of plasma derived profibrinolytic proteins, including urokinase-type plasminogen activator (u PA) and plasminogen (Fay, W. P et al, 1994 cited above; Lenich, C et al, 1997 Blood. 90:3579-3586; Jiang, Y et al. 1996 Blood 87:2775-2781; Holt, J. C., and Niewiarowski, S. 1980 Circulation 62:342a). However, these proteins are found at very low levels, and their activity is overwhelmed by the large amounts of PAI-1, which helps to stabilize nascent thrombi.

Recently, the effect of changing this balance in platelet fibrinolytic proteins has been described. Quebec Platelet Disorder (QPD) is a rare bleeding disorder not responsive to platelet transfusion, but responsive to anti-fibrinolytic agents, such as tranexamic acid (Hayward, C. P. et al, 1997 Blood 89:1243-1253; Hayward, C. P. et al, 1996 Blood 87:4967-4978; Hayward, C. P. et al, 1997 Brit. J. Haematol. 97:497-503). The etiology of QPD has been ascribed recently to ectopic expression of an excess of urokinase-type plasminogen activator in megakaryocytes and platelets (Kahr, W. H. et al., 2001 Blood 98:257-265). QPD platelets contain predominantly activated two-chain urokinase (tcu-PA). The etiology for the bleeding diathesis may in part be due to local release of activated urokinase-type plasminogen activator within thrombi leading to premature lysis. However, degradation of multiple platelet alpha-granular proteins, including vWF and Factor V, presumably by plasmin generated as a result of urokinase, may interfere with thrombus development as well.

There remains a need in the art for methods for harnessing the cellular mechanisms of hematopoietic secretory cells, such as platelets and other cells differentiated from hematopoietic progenitor cells, to enable these cells to deliver small molecules for therapeutic, diagnostic and research purposes.

### SUMMARY OF THE INVENTION

In one aspect, the invention provides a first composition comprising a therapeutic peptide or protein, a transport moiety capable of transporting the first peptide or protein into a hematopoietic cell, and an optional linker between the first protein and the transport moiety, the linker susceptible to cleavage by an intracellular enzyme in the platelet.

In yet another aspect, the invention provides a hematopoietic cell differentiated from a common myeloid progenitor cell, e.g., a platelet, containing a first composition, as described herein.

In still another aspect, the invention provides a second composition comprising multiple hematopoietic cells as described above.

In still a further aspect, the invention provides a method for generating a cell or a collection of hematopoietic cells derived from a common myeloid progenitor capable of delivering an above-described first composition to a mammalian patient comprising the step of transferring the first composition into the cell by contacting the cell or a collection of the cells with multiple copies of the first composition for sufficient time to permit the first composition to be transported into the cells.

In yet another aspect, the invention provides methods for treating or preventing certain disorders, diseases, symptoms or injuries in which cells of the hematopoietic lineage are involved, by delivering to a mammalian patient a suitable cell that contains, and is able to secrete, the therapeutic peptide or protein of the first composition described above. In one embodiment, such a disorder involves lung injury. In another embodiment, such a disorder includes a stroke, atherosclerosis or other cardiac disease.

In still a further aspect, the invention provides a method for preventing unwanted thrombus formation in a mammal by administering a platelet or collection of platelets containing the above-described composition, in which the first peptide or protein is a fibrinolytic protein. The platelet secretes the fibrinolytic protein at the site of the thrombus formation.

### BRIEF DESCRIPTION OF THE FIGURES

FIG. 1A is a schematic diagram illustrating the construction and expression of an exemplary first composition of the present invention, i.e., a recombinant fusion protein formed by fusion of a transport protein, e.g., HIV-1 TAT, fused through a cleavable linker, e.g., the sequence RKRRKR (SEQ ID NO: 1), to a therapeutic peptide or protein, e.g., the fibrinolytic protein, urokinase-type plasminogen activator (u-PA). The linker can be cleaved by the endoplasmic reticulum protease, BiP (NCBI database accession No. P20029; also known as GRP78) as well as a number of serine proteases.

FIG. 1B is a schematic illustrating the recombinant protein TAT-u-PA.

FIG. 2 is a bar graph indicating the bronchial alveolar lavage (BAL) protein concentration (mg/ml) in the mouse lungs over time in mice subjected to the mouse hyperoxia model of Example 5. The asterisk in the figure indicates that the results were statistically significant with a probability value of p<0.05 vs. baseline.

FIG. 3 is a bar graph indicating leukocyte kinetics in BAL in mice subjected to the mouse hyperoxia model of Example 5, with the light bars indicating white blood cells/ml BAL and the dark bars indicated polymorphonuclear (PMN) cells/ml BAL. Cell numbers are indicated at value×10³/ml BAL. The asterisk in the figure indicates that the results were statistically significant with a probability value of p<0.05 vs. baseline.

FIG. 4 is a graph showing survival times in hyperoxia according to the mouse model of Example 5, for control mice (not subjected to hyperoxia and indicated by the horizontal line at 100% survival), WT mice (subjected to 72 hours of hyperoxia, and indicated by the stepwise vertical line showing 0% survival at about 120 hours) and u-PA⁺ mice (subjected to 72 hours of hyperoxia, and indicated by the stepwise vertical line showing 0% survival at about 170 hours).

FIG. 5 is a bar graph depicting BAL protein concentration for the same groups of mice as discussed in FIG. 4, namely control mice (n=3), WT mice (n=8) and u-PA⁺mice (n=6) exposed to 100% O₂ for 72 hours. The asterisk indicates statistical significance at a probability of p<0.05 vs. WT.

FIG. 6 is a bar graph depicting BAL WBC counts for the same groups of mice, namely control mice (n=3), WT mice (n=10) and u-PA⁺ mice (n=8) exposed to 100% O₂ as in FIG. 4. The asterisk indicates statistical significance at a probability of p<0.05 vs. WT.

FIG. 7A is a bar graph, which plots % of brain area affected by a carotid artery infarct vs. “slice” number, showing the extent of the infarcted area in serial MRI images for a WT mouse in the stroke model of Example 6.

FIG. 7B is a bar graph, which plots % of brain area affected by a carotid artery infarct vs. “slice” number, showing the extent of the infarcted area in serial MRI images for a mUK (also known as u-PA⁺) mouse used in the stroke model of Example 6.

FIG. 8A is a graph of results from the carotid artery injury model of Example 7, showing data from one mouse injected with platelets pre-incubated with media from D. melanogaster S2 cells transfected with TAT-u-PA after induction with copper and one mouse injected with platelets pre-incubated with these S2 cells prior to induction with copper. Thrombosis score 0 represents no clot formation over a 30 minute observation; a score of 1 represents an unstable clot formation; and a score of 2 represents stable clot formation. Platelets pre-incubated with induced media containing TAT-u-PA protected against formation of stable occlusive carotid artery thrombosis. Platelets pre-incubated with uninduced media (and thereby containing no TAT-u-PA) had no effect upon the formation of stable clots.

FIG. 8B is a graph plotting thrombosis score in such an assay in which a mouse was injected with a sample of a pellet containing platelets preincubated with TAT-u-PA-containing induced media (5 μL) or a pellet containing platelets preincubated with uninduced media (containing no TAT-u-PA; 5 μL) or supernatant from the pelleted “uninduced” platelets (50 μL) or supernatant from the pelleted TAT-u-PA-containing induced media at concentrations of 50 μL or 5 μL, prior to ferric chloride injury as described above. As shown by the graph, only the pellet containing platelets preincubated with TAT-u-PA containing induced media (5 μL) was thrombolytic. Approximately half of the TAT-u-PA was found in supernatant (e.g., soluble) under these pelleting conditions, and the rest was cell-associated in the pellet. Soluble TAT-u-PA had no effect on the subsequent formation of stable occlusive thrombi, likely because of the rapid (1-2 minute) clearance of u-PA from the blood. In contrast, the pelleted, platelet-associated TAT-u-PA afforded total protection against carotid artery occlusion.

### DETAILED DESCRIPTION OF THE INVENTION

The present invention provides novel compositions for the transport of therapeutic peptides into cells of the hematopoietic lineage (particularly platelets, among other cells), collections of multiples of such cells which can deliver and release, but do not express, such therapeutic peptides, and methods for delivery of such therapeutic peptides in vivo by secretion from the cells. Methods for treatment and prophylaxis of various disorders using these cells are disclosed, particularly for disorders involving or mediated by cells of the hematopoietic lineage. Such disorders can include inflammations, infections, tissue injuries, vascular injuries, tumor growth, fibrosis and wound healing.

### I. COMPOSITION OF THE INVENTION

In one embodiment of this invention, a composition is provided that comprises a therapeutic peptide or protein, a transport moiety capable of transporting the therapeutic peptide or protein into a hematopoietic cell, and an optional linker between the therapeutic peptide or protein and the transport moiety. This latter component of the composition is susceptible to cleavage by an intracellular enzyme or other conventional peptide/protein cleaving mechanisms.

A. Definitions

As used herein, the term “amino acid” is used in its broadest sense, and includes naturally occurring amino acids as well as non-naturally occurring amino acids, including amino acid analogs and derivatives. “Naturally-occurring amino acid” is used herein to refer to the twenty amino acids that occur in nature in L form, which include alanine, cysteine, aspartate, glutamate, phenylalanine, glycine, histidine, isoleucine; lysine, leucine, methionine, aspargine, proline, glutamine, arginine, serine, threonine, valine, tryptophan, and tyrosine, or any derivative thereof produced through a naturally-occurring biological process or pathway.

“Non-naturally-occurring amino acid” is used herein to refer to an amino acid other than a naturally-occurring amino acid as defined above, which can be synthesized or “man-made”, and including a derivative thereof, whether produced synthetically or via a biological process or pathway. Non-naturally occurring amino acids include, without limitation, D amino acids, amino acids containing unnaturally substituted side chains, e.g., methyl-Arg, cyclic amino acids, diamino acids, β-amino acids, homo amino acids. Non-naturally-occurring or unnatural amino acids may be characterized by novel backbone and side chain structures and are widely available from commercial reagent suppliers, such as Sigma-Aldrich (www.sigmaaldrich.com), www.Netchem.com and other sites. See also a broad literature on such structures including, without limitation, Han S and Viola R E, Protein Pept. Lett. 2004 11(2):104-14; Ishida et al, Biopolymers 2004 76(1):69-82; Sasaki et al, Biol. Pharm. Bull. 2004 27(2):244-7; Pascal R et al, Meth. Enzymol. 2003 369:182-94; Yoder N C and Kumar K, Chem. Soc. Rev. 2002 31(6):335-41; and Ager D J, Curr. Opin. Drug Discov. Devel. 2002 5(6):892-905, among others, which are incorporated herein by reference. This term does not encompass those derivatives which fall within the definition of a “naturally-occurring amino acid”, as defined above. For example, one class of non-naturally occurring amino acids are L amino acids that effect stereochemistry. Thus, in one embodiment of compounds of this invention, one or more of the amino acids in the peptide may be in L form, while others may be in D form. Chemically synthesized compounds having properties known in the art to be characteristic of amino acids are also included in this definition.

Another non-naturally occurring amino acid is an amino acid which is modified to contain a substitution on the alpha-carbon in the amino acid structure. For example the alpha-carbon may be substituted by a suitable hydrocarbon moiety, such as aminoisobutyrate. Still another class of non-naturally occurring amino acids is amino acids which are modified or mutated to extend their carbon chain length. For example, an amino acid with a single alpha-carbon chain, may be extended with at least one additional carbon, i.e., a beta-carbon, and so on. An additional modification to an amino acid is the insertion of a substituent on the nitrogen of the amino group. An example of this type of modification is an N-methyl amino acid. The addition of substituents on the alpha carbon or additional carbons or on the nitrogen of the amino acid molecule may occur in any of the amino acids of the formula above.

Among useful substituents for creating the non-naturally occurring amino acids are a straight chain, branched, cyclic or heterocyclic C₁₋₁₂ alkyl group, and straight chain, branched, cyclic, or heterocyclic C₁₋₁₂ alkanoyl group. The amino acid may be also modified by the insertion of modifying sugars, imide groups and the like. Other amino acids are substituted in the ortho or meta position by a substituent such as H, OH, CH₃, halogen, OCH₃, NH₂, CH or NO₂.

A non-exclusive list of modified or non-naturally occurring amino acids for inclusion in components of the composition described herein include amino acids modified by N-terminal acetylation, C-terminal amidation, formylation of the N-terminal methionine, gamma-carboxyglutamic acid hydroxylation of Asp, Asn, Pro or Lys residues in the compound, methylation of Lys or Arg, preferably; phosphorylation of Ser, Thr, Tyr, Asp or H is in the compound, use of a pyrrolidone carboxylic acid, which is an N-terminal glutamate which has formed an internal cyclic lactam, sulfatation of Tyr, generally. Still other modifications of non-naturally occurring amino acids include use of or substitution with the following moieties: a 2-aminoadipic acid group, a 3-aminoadipic acid group, beta-Ala or beta-aminopropionic acid group, 2-aminobutryic acid, 4-aminobutyric acid, piperidinic acid, 6-aminocaproic acid, 2-aminoheptanoic acid, 2-aminoisobutryic acid, 3-aminoisobutyric acid, 2-aminopimelic acid, 2,4 diaminobutyric acid, desmosine, 2,2′-diaminopimelic acid, 2,3-diaminopropionic acid, N-ethylglycine, N-ethylglycine, N ethyl asparagine, hydroxylysine, allo-hydroxylysine, 3-hydroxyproline, 4-hydroxyproline, isodesmosine, allo-isoleucine, N-methylglycine, sarcosine, N-methylisoleucine, 6-N-methyllysine, N-methylvaline, 6-N-methyllysine, norvaline, norleucine, and ornithine.

As used herein, the term “proteogenic” indicates that the amino acid can be incorporated into a peptide, polypeptide, or protein in a cell through a metabolic pathway.

One of skill in the art may readily select among such non-naturally-occurring amino acids to prepare the therapeutic protein or peptide or the transport moiety or the linker components of the present composition to provide the desired characteristics useful in this invention. Such non-naturally occurring amino acid(s) when employed in the compounds above are anticipated to make the compounds more resistant to degradation by mammalian enzymes in serum, saliva, stomach and intestines, and thus compounds that are composed of one or more such amino acids may confer upon the compound enhanced stability and bioavailability in vivo. The incorporation of non-natural amino acids into the components of the compositions of the present invention is further desirable in circumstances in which increased stability or resistance to endogenous peptidases and proteases, improved bioavailability, prolonged intravascular and interstitial lifetimes and/or decreased immunogenicity are desirable. A variety of methods for producing non-natural amino acids are known and may be selected by one of skill in the art.

The term “homologous” as used herein, refers to the sequence similarity between two polymeric molecules, e.g., between two polypeptide molecules. When an amino acid position in both of the two molecules is occupied by the same monomeric amino acid, then they are homologous at that position. The homology between two sequences is a direct function of the number of matching or homologous positions, e.g., if half (e.g., five positions in a polymer ten subunits in length) of the positions in two compound sequences are homologous, then the two sequences are 50% homologous. If 90% of the positions, e.g., 9 of 10, are matched or homologous, the two sequences share 90% homology. By the term “substantially homologous” as used herein, is meant a peptide/polypeptide which is about 70% homologous, more preferably about 80% homologous, and most preferably about 90% homologous to the reference peptide/polypeptide.

Where, as discussed herein, proteins or compositions described herein are defined by their percent homologies or identities to identified sequences, the algorithms used to calculate the percent homologies or percent identities include the following: the Smith-Waterman algorithm (J. F. Collins et al, 1988, Comput. Appl. Biosci., 4:67-72; J. F. Collins et al, Molecular Sequence Comparison and Alignment, (M. J. Bishop et al, eds.) In Practical Approach Series: Nucleic Acid and Protein Sequence Analysis XVIII, IRL Press: Oxford, England, UK (1987) pp. 417), and the BLAST and FASTA programs (E. G. Shpaer et al, 1996, Genomics, 38:179-191). These references are incorporated herein by reference.

Such homologous sequences for the components of the compositions of this invention may be the result of modifications in the amino acid sequence of a peptide, polypeptide, or protein that provide improved, second generation peptides, etc., that display equivalent or superior functional characteristics when compared to the original amino acid sequence. Modifications include, without limitation, amino acid insertions, deletions, substitutions, truncations, fusions, shuffling of subunit sequences, changes in secondary structure of the sequence, and the like, provided that the peptide sequences produced by such modifications have substantially the same functional properties as the naturally occurring counterpart sequences disclosed herein. For example, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within +/−2 are substituted for one another. Like amino acids can also be substituted on the basis of hydrophilicity (see e.g., U.S. Pat. No. 4,554,101). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydropathic indices within +/2 are preferably substituted for one another.

B. Therapeutic Peptide/Protein

Depending upon the use for which the composition is constructed, the therapeutic peptide/protein is any peptide, polypeptide or protein useful for the treatment of a disorder or reduction or prevention of a symptom in which cells of the hematopoietic system are involved. For example, a non-exclusive list of therapeutic first peptide/proteins of this invention are useful for the treatment of a variety of inflammatory conditions, microbial or parasitic infections, injuries, such as vascular injuries and other hematopoietic cell-involved disorders. In one embodiment, such peptides/proteins include fibrinolytic proteins, including without limitation, urokinase-type plasminogen activator (u-PA), and tissue plasminogen activator (tpA). In another embodiment, the first peptide or protein is a procoagulant protein, such as Factor VIIa, Factor VIII, Factor IX and fibrinogen. Still other suitable proteins include plasminogen activator inhibitor-1 (PAI-1), von Willebrand factor, Factor V, ADAMTS-13 and plasminogen for use in altering the hemostatic balance at sites of thrombosis. Modified forms of these proteins, including forms with amino acid substitutions, deletions and chimerics are also useful.

Suitable first peptide/proteins also include, without limitation, hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful first peptide/proteins of this invention include proteins that regulate the immune system including, without limitation, cytokines and lymphokines such as thrombopoietin (TPO), interleukins (IL) IL-1 through IL-25 (including, IL-2, IL-4, IL-12, and IL-18), monocyte chemoattractant protein, leukemia inhibitory factor, granulocyte-macrophage colony stimulating factor, Fas ligand, tumor necrosis factors α and β, interferons α, β, γ, stem cell factor, and flk-2/flt3 ligand.

Peptides/proteins normally produced by the immune system are also useful in the invention. These include, without limitation, immunoglobulins IgG, IgM, IgA, IgD and IgE, chimeric immunoglobulins, humanized antibodies, single chain antibodies, T cell receptors, chimeric T cell receptors, single chain T cell receptors, class I and class H MHC molecules, as well as engineered immunoglobulins and MHC molecules. First peptide/proteins also include complement regulatory proteins, membrane cofactor protein (MCP), decay accelerating factor (DAF), CR1, CF2 and CD59.

Still other useful first peptide/proteins include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. The invention also encompasses first peptide/protein members of the steroid hormone receptor superfamily including glucocorticoid receptors and estrogen receptors, Vitamin D receptors and other nuclear receptors. In addition, useful first peptide/proteins include transcription factors such as jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, HNF4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful peptide/proteins include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence.

Still other useful first peptide/proteins for use in the compositions of this invention include enzymes such as are useful in enzyme replacement therapy, which composition is useful in a variety of conditions resulting from deficient activity of enzyme.

Further useful first peptide/proteins for the compositions of this invention include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients.

Compositions of this invention are employed to provide as first peptide/protein of this invention polypeptides that can reduce the activity or inactivate oncogenes such as myb, myc, fyn, and the translocation gene bcr/ab1, ras, src, P53, neu, trk and EGRF. Anti-cancer treatments and protective regimens include first peptide/proteins directed to inactivate or reduce the activity of variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides, which are found at higher levels in tumor cells including the polypeptide recognized by monoclonal antibody 17-1A and folate binding polypeptides.

Other suitable therapeutic polypeptides and proteins for use in the compositions of this invention include those which are useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Still other suitable first peptide/proteins of this inventions are fibrinolytic proteins and peptides suitable for delivery by compositions of this invention in platelets, such as illustrated in the below-noted examples.

The selection of the first peptide/protein of this invention is not a limitation of this invention. Choice of a first peptide/protein of this invention is within the skill of the artisan in accordance with the teachings of this application.

C. Transport Moiety

Useful transport components for the composition of this invention are peptides or proteins or other moieties that can translocate across the cell or plasma membrane and carry a fused protein into the cell. Preferably such transport moieties transfer the desired peptide or protein into the cell without activating it. However, in some circumstances, it may be desirable for the composition of the invention to initiate activation upon transfer.

Various methods have been developed recently for delivering macromolecules into cells in vitro. Known methods and components for delivery of biologically active proteins into intact cells in vitro are useful as components of the present invention.

Suitable embodiments of a transport moiety useful as a component of the present invention are “cell penetrating peptides” (CPP) or “protein transduction domains” (PTD). Suitable CPPs are arginine-rich peptides. More specifically, linear or branched-chain peptides containing approximately 8 residues of arginine are useful CPPs (See, e.g., Futaki et al 2003 Curr. Prot. Pept. Sci., 4(2):87-96; and Futaki 2002 Int. J. Pharm, 245(1-2): 1-7, both incorporated by reference herein). Other suitable CPPs are also discussed in International Published Patent Application Nos. WO 03/035892 and WO 03/035697.

Suitable PTDs include transactivating protein analogs or fragments thereof, such as the HIV-1 Tat (Vives et al, 2003 Curr. Protein Pept. Sci., 4(2): 125-32). The HIV-1 Tat basic peptide sequence is an example of the prototypic cell membrane-permeant component. U.S. Pat. No. 6,348,185 refers to cell membrane-permeant peptides including peptides of 4 to 6 amino acids derived from HIV-1 Tat, linked to pharmaceutically active substances via a functional linker that confers target cell specificity to the composition. U.S. Pat. Nos. 5,804,604; 5,747,641; 5,674,980; 5,670,617; 5,652,122 (Frankel) refers to the use of Tat peptides to transport covalently linked biologically active cargo molecules into the cytoplasm and nuclei of cells. Morris et al, 2001 Nat. Biotechnol., 19(12): 1173-76 refers to PTDs including TAT protein sequences. U.S. Pat. No. 5,804,604 refers to Tat-derived transport polypeptides. A commercial useful peptide transport molecule is the CHARIOT™ reagent (Active Motif)

Still other options for the transport moiety useful in the present invention are described in U.S. Pat. Nos. 5,135,736 and 5,169,933 (Anderson), which refer to the use of covalently linked complexes (CLCs) to introduce molecules into cells. CLCs comprise a targeting protein, preferably an antibody, a cytotoxic agent, and an enhancing moiety. Specificity is imparted to the CLC by means of the targeting protein, which binds to the surface of the target cell. After binding, the CLC is taken into the cell by endocytosis and released from the endosome into the cytoplasm. In one embodiment, Anderson refers to the Tat protein as part of the enhancing moiety to promote translocation of the CLC from the endosome to the cytoplasm. The complexes are limited in their specificity to cells that can be identified by cell surface markers. In addition, the attachment of enhancing moieties to the CLC is accomplished by the use of bifunctional linkers. The use of bifunctional linkers results in the production of a heterogeneous population of CLCs with varying numbers of enhancing moieties attached at varying locations.

Yet another embodiment of a transport moiety is the peptide-oligodeoxynucleotide conjugates described by L. Chaloin et al, 1997 Biochem., 37:11179-87. These conjugates comprise the combination of a peptide containing a hydrophobic motif associated with a hydrophilic nuclear localization sequence covalently linked to a small molecule to facilitate the cellular internalization of small molecules. The hydrophobic sequences used correspond to a signal peptide sequence or a fragment of the fusion peptide GP41. One peptide successfully targeted fluorescent oligodeoxynucleotides into living cells (Chaloin et al, 1998 Biochem. Biophys. Res. Commun., 243(2):601-608).

Still another transport peptide is described by Taylor et al, 2003 Electrophoresis, 24(9):1331-1337 and refers to an amphipathic peptide Pep-1 which may be used as a transport peptide in combination with a nonionic detergent carrier, for delivery of SDS-PAGE isolated proteins into a cell.

The transport moiety useful in the present invention can be any cell membrane-permeant basic peptide component of the complexes described in the above-cited documents, all of which are incorporated by reference herein. The transport moiety can be a peptide or protein that comprises any amino acid sequence (including naturally-occurring amino acids or non-natural amino acids, such as D amino acids) that confers the desired intracellular translocation and targeting properties to the selected therapeutic peptide or protein. Preferably, these amino acid sequences are characterized by their ability to confer transmembrane translocation and internalization of a complex construct when administered to the external surface of an intact cell of hematopoietic lineage. Attachment of the therapeutic protein/peptide to the transport moiety would permit the resulting composition to be localized within cytoplasmic and/or nuclear compartments. Specific PTDs or cell membrane-permeant peptide sequences useful in practicing the present invention include, but are not limited to, sequences of the following proteins and fragments and homologous sequences derived therefrom: the HIV-1 Tat protein, the HIV-1 Rev protein basic motif, the HTLV-1 Rex protein basic motif, the third helix of the homeodomain of Antennapedia, a peptide derivable from the heavy chain variable region of an anti-DNA monoclonal antibody, the Herpes simplex virus VP22 protein, the Chariot™ protein, and the Pep-1 protein. The minimum number of amino acid residues can be in the range of from about three to about six, preferably from about three to about five, and most preferably about four.

Proper transport and localization is demonstrated by a variety of detection methods such as, for example, fluorescence microscopy, confocal microscopy, electron microscopy, autoradiography, or immunohistochemistry.

Alternative transport moieties can include other methods and materials that have also been employed for delivery of proteins. Such methods of protein delivery into a cell include scrape loading, calcium phosphate precipitates, liposomes, electroporation, membrane fusion with liposomes, high velocity bombardment with DNA-coated microprojectiles, incubation with calcium-phosphate-DNA precipitate, DEAE-dextran mediated transfection, and direct micro-injection into single cells. Chemical addition of a lipopeptide (P. Hoffmann et al., 1988 Immunobiol., 177, pp. 158-70) or a basic polymer such as polylysine or polyarginine (W.-C. Chen et al., 1978 Proc. Natl. Acad. Sci. USA, 75, pp. 1872-76). Folic acid has been used as a transport moiety (C. P. Leamon and Low, 1991 Proc. Natl. Acad. Sci. USA, 88, pp. 5572-76). Pseudomonas exotoxin has also been used as a transport moiety (T. I. Prior et al., 1991 Cell, 64, pp. 1017-23).

As one example, Pro-Ject™ protein transfection reagent (Pierce Chem. Co.) uses a unique cationic lipid formulation that is non-cytotoxic and is capable of delivering a variety of proteins into numerous cell types. The first peptide of interest may be formulated with this transfection reagent for transport into a selected hematopoietic cell, e.g., a platelet, according to this invention.

Such methods may be substituted for the peptide/protein transport moiety, if desirable.

D. Optional Cleavable Linker

Still another component of the composition of this invention is an optional linker sequence that associates the therapeutic peptide/protein with the transport moiety. In one embodiment, such a linker sequence involves a sequence of amino acids susceptible to cleavage by native enzymatic activities in the hematopoietic cell, such as proteases, kinases, and phosphatases. In one embodiment, the intracellular enzyme is an endoplasmic reticulum protease, such as BiP. In another embodiment the intracellular enzyme is a serine protease, such as plasmin. The linker region can comprise amino acid residues, or substituted or unsubstituted hydrocarbon chains useful for connecting the transport moiety and the therapeutic protein, for example, via peptide bonds. Useful linker regions include, without limitation, natural and unnatural biopolymers. Non-exclusive examples of natural linkers include L-oligopeptides, while examples of unnatural linkers are D-oligopeptides, lipid oligomers, liposaccharide oligomers, peptide nucleic acid oligomers, polylactate, polyethylene glycol, cyclodextrin, polymethacrylate, gelatin, and oligourea (Schilsky, et al., Eds., Principles of Antineoplastic Drug Development and Pharmacology, Marcel Dekker, Inc., New York, 1996, pp. 741).

Exemplary linker sequences comprise protein kinase consensus sequences, protein phosphatase consensus sequences, or protease-reactive or protease-specific sequences. Protease sequences are particularly desirable. Additional examples of cleavable linkers useful in this invention include recognition motifs of exo- and endo-peptidases, extracellular metalloproteases, lysosomal proteases such as the cathepsins (cathepsin B), HIV proteases, as well as transferases, hydrolases, isomerases, ligases, oxidoreductases, esterases, glycosidases, phospholipases, endonucleases, ribonucleases, beta-lactamases and metalloproteinases.

Essentially any amino acid sequence that serves to link the two other components of the composition and can be cleaved inside the hematopoietic cell to release the therapeutic peptide is useful in this aspect of the invention. Many such sequences are known in the art and several are disclosed in the above-recited documents incorporated by reference herein.

E. Miscellaneous Components of the Composition

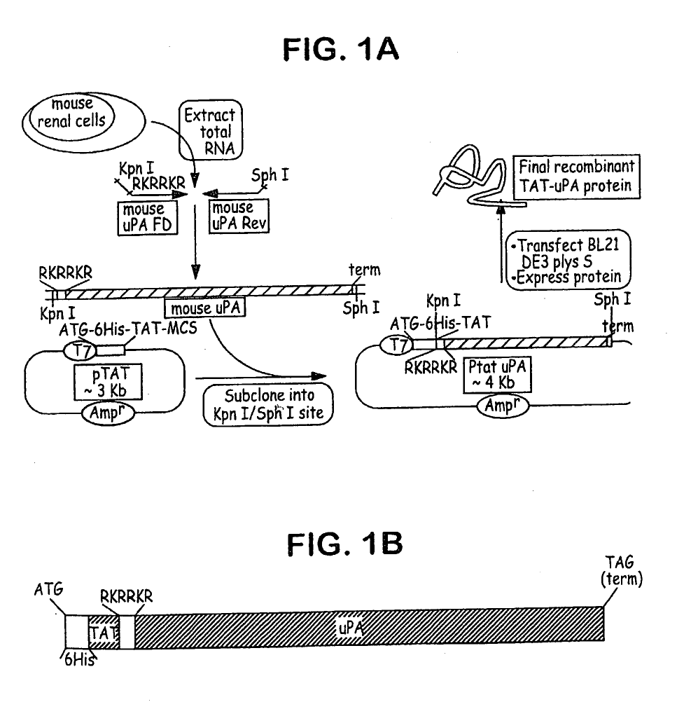

Other components of the composition of this invention include other amino acid or synthetic chemical compounds introduced into or adjacent to the therapeutic peptide or transport moiety for the purpose of enhancing purification. For example, a poly-histidine sequence inserted at the amino terminus of the composition is frequently useful for purification of recombinant protein constructs. Such a sequence appears in the exemplary construct illustrated in the examples below and in FIGS. 1A and 1B. Still other additional components of the composition include the Herpes virus structural peptide VP22 or the positive domain of the Drosophila homeotic transcript factor. These sequences are also introduced into or adjacent to the therapeutic peptide or transport moiety for the purpose of enhancing purification or transport.

F. Methods of Making the Composition

The compositions of this invention that comprise the therapeutic peptide, transport moiety and optional linker may be prepared recombinantly or by other suitable methods, such as recombinant methods, synthetic methods, e.g., mutagenesis, or combinations of such methods. The sequences or molecules of this invention are not limited solely to the specific peptides, linkers and transport sequences presented herein, but rather include any and all sequences that share homology (i.e., have sequence identity) or function with the sequences presented herein. The preparation or synthesis of the compositions of this invention is well within the ability of the person having ordinary skill in the art using available material.

As one example, the compositions of this invention, or portions thereof, may be produced by chemical synthesis methods. For example, the nucleotide sequences useful in the invention are prepared conventionally by resort to known chemical synthesis techniques, e.g., solid-phase chemical synthesis, such as described by Merrifield, 1963 J. Amer. Chem. Soc., 85:2149-2154; J. Stuart and J. Young, Solid Phase Peptide Synthesis, Pierce Chemical Company, Rockford, Ill. (1984); Matteucci et al., 1981 J. Am. Chem. Soc., 103:3185; Alvarado-Urbina et al., 1980 Science, 214:270; and Sinha, N. D. et al., 1984 Nucl. Acids Res., 13:4539, among others. See, also, e.g., PROTEINS—STRUCTURE AND MOLECULAR PROPERTIES, 2nd Ed., T. E. Creighton, W.H. Freeman and Company, New York, 1993; Wold, F., “Posttranslational Protein Modifications: Perspectives and Prospects”, pgs. 1-12 in POSTTRANSLATIONAL COVALENT MODIFICATION OF PROTEINS, B.C. Johnson, Ed., Academic Press, New York, 1983; Seifter et al., 1990 Meth. Enzymol., 182:626-646, and Rattan et al., 1992 Ann. N.Y. Acad. Sci., 663:48-62.

Alternatively, the molecules of this invention are constructed recombinantly using conventional molecular biology techniques, site-directed mutagenesis, genetic engineering or polymerase chain reaction, such as, by cloning and expressing a nucleotide molecule encoding a desired therapeutic protein with optional other proteins within a host cell utilizing the information provided herein (See, e.g., Sambrook et al., cited above; Ausubel et al. cited above). Coding sequences for the first peptide/protein, the linker and the transport moiety of this invention can be prepared synthetically (W. P. C. Stemmer et al. 1995 Gene, 164:49).

Transport peptide/proteins of this invention may be advantageously attached to the therapeutic peptide/proteins and the linkers by chemical cross-linking or by genetic fusion. The attachment of the therapeutic peptide/protein of interest to a transport peptide/protein to produce a composition of this invention may be effected by any means which produces a link between the two constituents which is sufficiently stable to withstand the conditions used and which does not alter the function of either constituent. Preferably, the link between them is covalent. For example, recombinant techniques can be used to covalently attach the transport protein to the therapeutic protein, such as by joining the gene coding for Tat with the gene coding for the therapeutic peptide and introducing the resulting gene construct into a cell capable of expressing the composition. Alternatively, the two separate nucleotide sequences can be expressed in a cell or can be synthesized chemically and subsequently joined, using known techniques. Alternatively, the first peptide/protein-transport moiety can be synthesized chemically as a single amino acid sequence (i.e., one in which both constituents are present) and, thus, joining is not needed. A unique terminal cysteine residue is a preferred means of chemical cross-linking. According to some preferred embodiments of this invention, the carboxy terminus of the transport moiety is genetically fused to the amino terminus of the cargo moiety via the linker. Numerous chemical cross-linking methods are known and potentially applicable for conjugating the transport polypeptides of this invention to the first peptide/protein. Many known chemical cross-linking methods are non-specific, i.e., they do not direct the point of coupling to any particular site on the transport moiety or therapeutic peptide. As a result, use of non-specific cross-linking agents may attack functional sites or sterically block active sites, rendering the conjugated proteins biologically inactive.

A preferred approach to increasing coupling specificity in the practice of this invention is direct chemical coupling to a functional group found only once or a few times in one or both of the polypeptides to be cross-linked. For example, in many proteins, cysteine, which is the only protein amino acid containing a thiol group, occurs only a few times. Also, for example, if a polypeptide contains no lysine residues, a cross-linking reagent specific for primary amines will be selective for the amino terminus of that polypeptide. Successful utilization of this approach to increase coupling specificity requires that the polypeptide have the suitably rare and reactive residues in areas of the molecule that may be altered without loss of the molecule's biological activity.

As demonstrated in the examples below, cysteine residues may be replaced when they occur in parts of a polypeptide sequence where their participation in a cross-linking reaction would likely interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine is preferred as a replacement for cysteine. As demonstrated in the examples below, a cysteine residue may be introduced into a polypeptide's amino acid sequence for cross-linking purposes. When a cysteine residue is introduced, introduction at or near the amino or carboxy terminus is preferred. Conventional methods are available for such amino acid sequence modifications, whether the polypeptide of interest is produced by chemical synthesis or expression of recombinant DNA.

Coupling of the two constituents can be accomplished via a coupling or conjugating agent. There are several intermolecular cross-linking reagents that can be utilized (see, for example, Means, G. E. and Feeney, R. E., Chemical Modification of Proteins, Holden-Day, 1974, pp. 3943). Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate, diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents may be homobifunctional, i.e., having two functional groups that undergo the same reaction. A preferred homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of polypeptides that contain cysteine residues.

Cross-linking reagents may also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (“SMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these cross-linkers reacts with a primary amine, and the thiol-reactive maleimide forms a covalent bond with the thiol of a cysteine residue.

Cross-linking reagents often have low solubility in water. A hydrophilic moiety, such as a sulfonate group, may be added to the cross-linking reagent to improve its water solubility. Sulfo-MBS and sulfo-SMCC are examples of cross-linking reagents modified for water solubility.

The cross-linking reagent, if used, should preferably contain a covalent bond, such as a disulfide, that is cleavable under cellular conditions. For example, dithiobis(succinimidylpropionate) (“DSP”), Traut's reagent and N-succinimidyl 3-(2-pyridyldithio) propionate (“SPDP”) are well-known cleavable cross-linkers. The use of a cleavable cross-linking reagent permits the therapeutic peptide to separate from the transport moiety after delivery into the hematopoietic cell. Direct disulfide linkage may also be useful.

Numerous cross-linking reagents, including the ones discussed above, are commercially available. Detailed instructions for their use are readily available from the commercial suppliers. A general reference on protein cross-linking and conjugate preparation is: S. S. Wong, Chemistry of Protein Conjugation and Cross-Linking, CRC Press (1991).

Chemical cross-linking may include the use of spacer arms. Spacer arms provide intramolecular flexibility or adjust intramolecular distances between conjugated moieties and thereby may help preserve biological activity. A spacer arm may be in the form of a polypeptide moiety comprising spacer amino acids. Alternatively, a spacer arm may be part of the cross-linking reagent, such as in “long-chain SPDP” (Pierce Chem. Co., Rockford, Ill., cat. No. 21651H).

The synthesis methods are not a limitation of this invention. The examples below detail presently preferred embodiments of synthesis of sequences encoding an exemplary composition of this invention. However, similar methods are employed to produce other recombinant molecules for the generation of therapeutic or prophylactic compositions of this invention.

G. Formulations

Preferably, the protein compositions of this invention are contained in a suitable physiologically acceptable diluent or a pharmaceutically acceptable carrier, such as sterile water or sterile isotonic saline prior to contact with the intended cell target. As used herein the language “pharmaceutically acceptable carrier” is intended to include any and all solvents, dispersion media, coatings, antibacterial and antifungal agents, isotonic and absorption delaying agents, and the like, compatible with administration to humans or other vertebrate hosts. The appropriate carrier will be evident to those skilled in the art and will depend in large part upon the route of administration.

Still additional components that are optionally present with the composition of this invention are adjuvants, preservatives, chemical stabilizers, or other proteins. Typically, stabilizers, adjuvants, and preservatives are optimized to determine the best formulation for efficacy in the target human or animal. Suitable exemplary preservatives include chlorobutanol, potassium sorbate, sorbic acid, sulfur dioxide, propyl gallate, the parabens, ethyl vanillin, glycerin, phenol, and parachlorophenol. Suitable stabilizing ingredients that are used include, for example, casamino acids, sucrose, gelatin, phenol red, N-Z amine, monopotassium diphosphate, lactose, lactalbumin hydrolysate, and dried milk.

Still other suitable optional components of the composition of this invention include, but are not limited to: surface active substances (e.g., hexadecylamine, octadecylamine, octadecyl amino acid esters, lysolecithin, dimethyl-dioctadecylammonium bromide), methoxyhexadecylglycerol, and pluronic polyols; polyamines, e.g., pyran, dextransulfate, poly IC, carbopol; peptides, e.g., muramyl dipeptide, dimethylglycine, tuftsin; oil emulsions; and mineral gels, e.g., aluminum phosphate, etc. and immune stimulating complexes, liposomes, polysaccharides, lipopolysaccharides and/or other polymers.

As exemplified below in the examples and in FIGS. 1A and 1B, a recombinant protein pTAT-u-PA was constructed as a composition of this invention for transport into platelets.

### II. CELL/COLLECTION OF CELLS

The cells or collection of cells containing the above-described protein compositions may be animal cells or human cells. More preferably, a hematopoietic progenitor cell or a cell differentiated therefrom is useful in the methods and compositions of the present invention. Hematopoietic stem cells are pluripotent cells present in the bone marrow, which divide to produce more specialized progenitor stem cells, i.e., lymphoid progenitors and myeloid progenitors. Cells that are differentiated from the lymphoid progenitors in the bone marrow and that are found in the peripheral blood include B cells and T cells. From B cells are generated plasma cells; from T cells are generated activated T cells.

The common myeloid progenitor stem cells produce the granulocytes/macrophage progenitor cells and the megakaryocyte/erthyrocyte progenitor cells in the bone marrow. Cells differentiated from the granulocyte/macrophage progenitors that are present in the blood and useful in the present invention include neutrophils, eosinophils, basophils, monocytes and immature dendritic cells. Still other suitable cells differentiated from monocytes are mast cells, macrophages and dendritic cells, which are present in tissue and lymph nodes. Cells differentiated from the megakaryocyte/erthrocyte progenitors include megakaryocytes and erythroblasts, which further differentiate into platelets and erythrocytes (red blood cells) in the blood. Suitable cells differentiated from the megakaryotic/erythrocyte progenitor cells are platelets, megakaryocytes or erythrocytes. Suitable cells differentiated from said lymphoid progenitors are natural killer cells.

Secretory cells of the hematopoietic lineage release the contents of their granules upon activation. Among such secretory cells are platelets, mast cells, neutrophils, eosinophils, etc. See, e.g., IMMUNOBIOLOGY, THE IMMUNE SYSTEM IN HEALTH AND DISEASE, 5^{th} edit., C. Janeway et al., Ed., Garland Publishing, New York, N.Y.: 2001.

In one embodiment, the protein-transport moiety-linker composition generated as described above is transferred as protein into a selected hematopoietic cell or collection of cells, as described above. The selected hematopoietic cells of the hematopoietic lineage are harvested from bone marrow of a suitable human or non-human mammal by conventional techniques. In one embodiment, the cells are isolated from a mammalian patient for autologous transfer, once the cells are contacted with the composition of the invention. Alternatively, the mammal providing the cells is a different mammal, preferably of the same species, for either introduction into another homologous mammal or for research or laboratory use. Methods for isolating such cells from bone marrow or peripheral blood are well known. See, for example, the Stem Cell Database of Princeton University; Phillips, R L et al, 2000 Science, 288:1635-1640 and references cited therein.

In another embodiment, cells of the hematopoietic lineage are those cells found in the peripheral blood or tissue, such as platelets, megakaryocytes, neutrophils, eosinophils, monocytes, basophils, dendritic cells, mast cells, macrophages, dendritic cells, erythrocytes, and natural killer cells. These cells must be isolated from the peripheral blood or tissue of a suitable human or non-human mammal. For example, the cells are isolated from a mammalian patient for autologous transfer. Alternatively, the mammal providing the host cells is a different homologous mammal, for either introduction into another mammal or for research or laboratory use. Methods for isolating such cells from peripheral blood or tissue are well known. For example, the platelets may be a heterologous collection of platelets obtained from a blood donor or donors, treated and collected as usual by the Red Cross or other blood collection agency. Alternatively, the platelet or platelets may be collected from the peripheral blood of the same subject to be treated for a disease condition.

In one particular embodiment the hematopoietic cell is a platelet or collection of platelets. The process is described below with reference to platelets for ease of discussion. However, it will be clear to one of skill in the art that the process may readily be adapted to some other hematopoietic cell.

The selected hematopoietic cell, e.g., platelet, isolated from an autologous or heterologous source, is contacted in vitro or ex vivo with a composition of the present invention under appropriate conditions to prepare the cell and collection of cells of this invention. Essentially, the contacting involves incubating the selected cells in a suitable buffer or saline containing the protein compositions as described above and in the Examples below for sufficient time to permit the composition to permeate the cells.

The amount of the protein composition to be added to the cells may be determined by the person of skill in the art with reference to the type of cells, the therapeutic protein selected, and the disease to be treated. Typically, an excess of composition is added to the collection of cells. One exemplary range for the amount or concentration of the protein composition to be added to a collection of cells is from 50 μg protein composition/cell to 50 mg of protein composition/cell. Stated in another way, the amount of protein to be added to the cells may range, in one embodiment, from 1 to 10 times the physiological concentration of the cells. Still other amounts may be determined by one of skill in the art, given the condition being treated and other considerations based on the patient and his/her physiological condition.

Culture conditions sufficient to permit the protein compositions to permeate the cells may include a temperature of from about 20° C. to about 50° C. In another embodiment, the culture conditions include a temperature of from about 30° C. to about 40° C. and, even more preferably about 37° C. The pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Typically, the cells are permitted to incubate in the presence of the protein composition of this invention for a period of at least 30 minutes. In other embodiments the contact time may range from 10 minutes to 2 hours. Still longer incubation periods may be desirable. The conditions for the contact between the hematopoietic cells and the selected composition of this invention may vary depending upon the identity of the cells, the number of cells being treated, and the dosage desired for treatment. The parameters of such conditions may readily be altered by one of skill in the art. See, e.g., the Examples below.

After a sufficient time has passed for maximal permeation of the protein composition into the cells, the hematopoietic cells may be optionally separated from any excess extracellular protein composition prior to use and/or storage. Suitable separation techniques include filtration, centrifugation, separation on a column, etc. The separation technique should be chosen which does not damage the cells. Platelets are routinely purified either by gel filtration, sedimentation, or centrifugation. Alternatively, the platelets may be infused without separation, e.g., as is, in the plasma. The cells or collection of cells are thereafter dispersed in a physiologically acceptable diluent or a pharmaceutically acceptable carrier, such as sterile water or sterile isotonic saline, having appropriate pH isotonicity, stability and other conventional characteristics for maintenance of cells of the type selected. As used herein the language “pharmaceutically acceptable carrier” is intended to include any and all solvents, dispersion media, coatings, antibacterial and antifungal agents, isotonic and absorption delaying agents, and the like, compatible with administration to humans or other vertebrate hosts.

Other components may be added to the collection of platelets or to the plasma, as is, before infusion. Such additional components may include, without limitation, apyrase, theophylline, or PGE1, which are added to prevent platelet activation.

The cell/cell collection of the present invention may be composed of activated cells containing the protein composition of the invention or non-activated proteins containing the protein composition of the invention. In some embodiments, it is preferred that the cells be non-activated following permeation by the protein compositions. In other embodiments, the activated state of the cells/cell collections of the invention is also useful. In still other embodiments, the cells are activated by in vivo conditions. For example, platelets may be activated by conditions associated with the presence of arterial injury. Any cells/cell collections of the invention may be lyophilized for storage. Lyophilization conditions would be typical for the type of cell in the cell collection. However, if not lyophilized, all such cells are preferably used within several hours following preparation.

Specific non-limiting examples of such cell/cell collections containing a therapeutic protein-transport moiety protein of this invention include the following: a collection of platelets, in which the therapeutic protein or peptide is Factor VIIa, Factor VIII, Factor IX or fibrinogen; or a collection of platelets in which the therapeutic protein or peptide is urokinase plasminogen activator, plasminogen, tissue plasminogen activator, or tissue factor pathway inhibitor. Another embodiment of this invention is an erythrocyte or collection of erythrocytes in which the therapeutic protein or peptide is urokinase plasminogen activator receptor. Still another embodiment of this invention is a neutrophil or collection of neutrophils, in which the therapeutic protein or peptide is activated Protein C. A further useful embodiment of this invention is an eosinophil or collection of eosinophils, in which the therapeutic protein or peptide is a protein or fragment thereof toxic to a helminth. Another embodiment of a cell/cell collection of this invention is an eosinophil or collection of eosinophils in which the therapeutic protein or peptide of the protein composition is human TSG6, an antibody to IL-1 receptor alpha or an anti-inflammatory protein. Still another embodiment of a cell/cell collection of this invention is an NK cell or collection of NK cells, in which the therapeutic protein or peptide is a neutralizing antibody against a viral coat protein, such as anti-HIV 1 gag protein or anti-HPV proteins.

Given this disclosure, one of skill in the art can readily prepare a variety of other cell/cell collections falling within the scope of the claims. The examples below discuss one embodiment of this invention, namely platelets incubated in the presence of an exemplary u-PA and Tat composition, used for delivery of the u-PA to the site of arterial injury. At the site of the arterial injury, the platelets are activated, and secrete the u-PA, which demonstrates thrombolytic function.

### III. METHODS OF USING THE CELL/CELL COLLECTIONS

Once selected cells of the hematopoietic lineage contain a suitable number of compositions of this invention, the cells or collections of cells are employed in pharmaceutical or prophylactic compositions and methods for the treatment of a variety of disorders in human or non-human mammals. Such treatment may include enhancement of a biological activity or reduction or a disadvantageous biological activity occurring in the body. Similarly, the cells are used in direct treatment of disorders such as inflammatory disorders, microbial or parasitic infection, vascular or hemorrhagic disorders, and the like in which the hematopoietic cells, their progenitors or differentiated cells are implicated. Specifically among such disorders are the treatment of injury to the lungs and the treatment of stroke.

Of particular significance is the use of cells, which are naturally secretory, such as platelets, to deliver the therapeutic protein to selected sites in the subject's body, e.g., sites of thrombus formation to prevent further progression of stable, occlusive thrombus development.

One method for treating a disorder in a mammal according to this invention includes the step of administering to a mammalian hematopoietic lineage cell containing a first peptide/protein-transport protein composition of this invention. Where the cell is secretory, the method permits a cell of a hematopoietic lineage, e.g. a platelet, to migrate to a suitable site in the mammal and secrete the selected first peptide/protein, which has been cleaved from the transport moiety by the operation of a naturally-occurring intracellular enzyme. For example, a platelet secreting a first peptide/protein of this invention will target to the site of vascular injury or thrombus formation.

In one embodiment, therefore, the method of the invention reinfusing autologous cells contacted as described above with the therapeutic protein/transfer moiety composition of this invention into the bone marrow or peripheral blood of the mammalian patient. Alternatively, the method of treatment can involve infusing or injecting into the patient's bone marrow or blood a non-self (heterologous) cell containing the first peptide/protein of this invention. In still other embodiments, the cells containing the first peptide/protein of this invention may be administered into the site of a wound, into the pleural or peritoneal space, or into a joint. In some embodiments, administration is alternatively intravenous or directly into the bone marrow. Other suitable routes of administration include, but are not limited to, intravenous and intraarterial. The appropriate route is selected depending on the nature of the cells used, the disease condition to be treated, and an evaluation of the age, weight, sex and general health of the patient and similar factors by an attending physician.

Suitable doses of cells or cell collections of this invention are readily determined by one of skill in the art, depending upon the condition being treated, the health, age and weight of the veterinary or human patient, and other related factors, and the other characteristics of the composition. In general, selection of the appropriate “effective amount” or dosage for the cells of the present invention will also be based upon the type of cell, the identity of the first peptide/protein of this invention and cell, as well as the physical condition of the subject. The method and routes of administration and the presence of additional components in the compositions may also affect the dosages and amounts of the compositions. Such selection and upward or downward adjustment of the effective dose is within the skill of the art. The amount of cells required to produce a suitable response in the patient to the therapeutic protein/peptide released by the cell without significant adverse side effects varies depending upon these factors. Suitable doses are readily determined by persons skilled in the art.

As an example, one “dosage” of platelets can be the equivalent of about 10% of the subject's platelet mass to permit inhibited thrombus development. It is contemplated that even greater percentages of platelet mass will be even higher percentages of total normal platelet mass. Similarly, where the cells are hematopoietic cells other than platelets, therapy can involve replacement of normal cell mass of 5%, 10%, 20% or more to achieve the therapeutic object.

As another example, in one embodiment, for the cells containing the therapeutic protein/transport moiety composition of this inventions, each dose may comprise sufficient numbers of cells in a mL to deliver between about 20 pg to about 20 mg of the therapeutic protein per mL. The number of transfused cells may range from about 0.1% to about 10% of the body's available pool, depending on the effectiveness of the stored protein. Other dosage ranges may also be contemplated by one of skill in the art. This dose is formulated in a pharmaceutical composition, as described above (e.g., suspended in about 0.01 mL to about 1 mL of a physiologically compatible carrier) and delivered by any suitable means.

The number of doses and the dosage regimen for the infusions of cells treated according to this invention are also readily determined by persons skilled in the art. The intended therapeutic or prophylactic effect is conferred by a single infusion of the cells or may require the administration of several doses or infusions, in addition to booster doses or infusions at later times.

The infusion of cells is repeated, as needed or desired, daily, weekly, monthly, or at other selected intervals.

One of skill in the art may readily identify a number of disorders that may be susceptible to treatment with the cells or cell collections of this invention, depending upon the selection of cell type and therapeutic peptide/protein in the composition. Among such disorders are included without limitation, coagulation disorders (either an insufficiency or excess thereof), acute lung injury and sepsis, helminth infection, asthma or other allergic reactions, viral infections, bacterial infections, etc.

For example, the compositions of this invention are useful in the prevention and/or treatment of disease(s) caused by microbial infections including, without limitation, Haemophilus influenzae (both typable and nontypable), Haemophilus somnus, Moraxella catarrhalis, Streptococcus pneumoniae, Streptococcus pyogenes, Streptococcus agalactiae, Streptococcus faecalis, Helicobacter pylori, Neisseria meningitidis, Neisseria gonorrhoeae, Chlamydia trachomatis, Chlamydia pneumoniae, Chlamydia psittaci, Bordetella pertussis, Alloiococcus otiditis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium-Mycobacterium intracellulare complex, Proteus mirabilis, Proteus vulgaris, Staphylococcus aureus, Staphylococcus epidermidis, Clostridium tetani, Leptospira interrogans, Borrelia burgdorferi, Pasteurella haemolytica, Pasteurella multocida, Actinobacillus pleuropneumoniae and Mycoplasma gallisepticum.

The compositions of this invention are useful in the prevention and/or treatment of disease caused by, without limitation, Respiratory syncytial virus, Parainfluenza virus types 1-3, Human metapneumovirus, Influenza virus, Herpes simplex virus, Human cytomegalovirus, Human immunodeficiency virus, Simian immunodeficiency virus, Hepatitis A virus, Hepatitis B virus, Hepatitis C virus, Human papillomavirus, Poliovirus, rotavirus, caliciviruses, Measles virus, Mumps virus, Rubella virus, adenovirus, rabies virus, canine distemper virus, rinderpest virus, avian pneumovirus (formerly turkey rhinotracheitis virus), Hendra virus, Nipah virus, coronavirus, parvovirus, infectious rhinotracheitis viruses, feline leukemia virus, feline infectious peritonitis virus, avian infectious bursal disease virus, Newcastle disease virus, Marek's disease virus, porcine respiratory and reproductive syndrome virus, equine arteritis virus and various Encephalitis viruses.

The compositions of this invention are useful in enhancing response against fungal pathogens such as Aspergillis, Blastomyces, Candida, Coccidiodes, Cryptococcus and Histoplasma or against parasites including Leishmania major, Ascaris, Trichuris, Giardia, Schistosoma, Cryptosporidium, Trichomonas, Toxoplasma gondii and Pneumocystis carinii.

Compositions of this invention may also be useful for the prevention and/or treatment of disease(s), without limitation, such as autoimmune disease, such as multiple sclerosis, lupus and rheumatoid arthritis and others, asthma, atherosclerosis, Alzheimer disease, amyloidosis or amyloidogenic disease, and cancers. Clotting disorders and other vascular injuries caused by other infections, injury, aging, thrombocytopenia, inappropriate thrombus formation, myelodysplasia, AML, stroke, atherosclerosis, and the like may also be treated according to the methods of this invention. These compositions and methods can be useful to treat allergic reactions to allergens such as pollen, insect venoms, animal dander, fungal spores and drugs (such as penicillin). Other conditions that are treated by the methods of this invention included disease characterized by unwanted thrombus formation, amyloid deposition, diabetes, and gastroesophageal reflux disease, among others. The methods of this invention may also be useful in the enhancement of wound healing. The selection of the disorder to be treated by the compositions and methods of this invention is not a limitation of this invention. One of skill in the art may readily include other disorders suitable for the treatment described herein.

Thus, as one specific embodiment, the invention provides a method for enhancing coagulation in a patient by delivering to the mammalian patient with an insufficient clotting mechanism a platelet (or other hematopoietic cell as described above) containing a first peptide/protein of this invention. Examples of suitable first peptide/protein of this invention for this method are first peptide/protein of this invention encoding one or more of Factor VIIa, Factor VIII, Factor IX or fibrinogen.

Another specific embodiment involves a method for preventing or reducing coagulation in a mammalian patient, where needed. According to this method, the patient is administered a platelet (or other hematopoietic cell as described above) containing a first peptide/protein of this invention. Examples of suitable first peptide/protein of this invention for this method are one or more of urokinase plasminogen activator, plasminogen, tissue plasminogen activator, and tissue factor pathway inhibitor.

Another example of a method of this invention is a method for enhancing coagulation in a mammalian patient by delivering to the patient a neutrophil or monocyte (or other hematopoietic cell as described above) secreting or producing a first peptide/protein of this invention. Examples of suitable first peptide/protein of this invention for this method are urokinase plasminogen activator receptor.

Still another example of this invention is a method for treating acute lung injury and sepsis in a mammalian patient. This method includes delivering to the patient a neutrophil (or other hematopoietic cell as described above) containing a first peptide/protein of this invention and secreting or producing same. Examples of a suitable first peptide/protein of this invention for this method is activated Protein C.

The methods and compositions of this invention may also be employed for treating parasitic helminth infection of a mammalian human or non-human patient. This method involves delivering to a mammal eosinophils (or other hematopoietic cell as described above) containing the first peptide/protein of this invention and secreting or producing same. An example of a suitable first peptide/protein is a protein toxic to a helminth.

Another method according to this invention involves treating asthma or allergic responses in a mammalian patient. This method involves delivering to a mammal an eosinophil (or other hematopoietic cell as described above) containing a first peptide/protein of this invention and secreting or producing same. Examples of suitable first peptide/proteins of this invention for this method are human TSG6, an antibody to IL-1 receptor alpha, and an anti-inflammatory protein.

The invention also includes a method for treating a viral infection in a mammal comprising delivering to a mammal an NK cell (or other hematopoietic cell as described above) containing a first peptide/protein of this invention. An example of a suitable first peptide/protein of this invention for this method is a neutralizing antibody against a viral coat protein, e.g., anti-HIV gag protein, anti-HPV proteins, anti-HIV proteins, etc.

Still another embodiment of a method of this invention is a method for the treatment and prevention of undesirable thrombus development in a mammalian patient by administering to the patient a platelet secreting urokinase-type plasminogen activator to control thrombus formation in a patient. See the Examples below.

### IV. KITS

The present invention also provides kits comprising a predetermined quantity of a therapeutic protein/peptide-linker-transport moiety composition of this invention for ready combination with a collection of suitable cells, e.g., such as a suitable Red Cross bag of platelets for transfusion typically provided to hospitals. The composition of this invention can be lyophilized to facilitate storage stability and contained in a sealed, sterilized container. Instructions for carrying out the process of introduction of the composition into a suitable collection of cells prior to use of the cells for infusion into a patient, as well as any necessary buffer solution(s), can also be included in the kit.

In one embodiment, the present invention provides a kit for use in preparing platelets for infusion into a patient for prevention of thrombus formation in the event of a stroke or heart attack. The kit can contain a lyophilized preparation of the rTat-u-PA composition of the Examples in sufficient dosage for combination with a conventional bag of platelets for infusion into the patient.

As reported in the following examples, an embodiment of the compositions and methods of this invention is demonstrated by using a composition comprising a therapeutic protein, e.g., u-PA, and a delivery sequence, e.g., TAT. This composition is incorporated as a protein into a collection of platelets, by incubation. Further these examples demonstrate that such platelets may then be used to deliver the u-PA to a site of injury.

### V. ADVANTAGES OF THE INVENTION

As discussed above, the cells of the present invention are not genetically engineered to express the therapeutic protein, as are cells used in gene therapy, but rather are manipulated to simply serve as a delivery mechanism. This invention provides advantages not provided by a method using cells which have been genetically modified to express a therapeutic protein. Particularly with respect to hematopoietic cells, such as platelets, megakaryocytes, etc., it is presently quite difficult to culture sufficient numbers of genetically engineered cells for use in gene therapy applications. In contrast, this invention employs existing collections of such cells, e.g., by Red Cross collection, and permits them to be subjected to a simply incubation to create the delivery vehicles. Thus the processes by which cells according to this invention are prepared may be readily reviewed by appropriate authorities for safety. Further, the amount of the therapeutic protein that will be delivered by the cells may be closely controlled because the cell can only deliver that amount of protein which was transferred into the cell during the incubation process. The cells, e.g., platelets, may be activated in vivo by physiological conditions existing at the site of arterial injury, so that the therapeutic protein is released at the site of a damaged vessel, not elsewhere in the body. Additionally, transport of the protein in the cell and release at the site of injury protects the protein from unwanted natural clearance, as would occur if the protein itself were administered directly into the circulation. For example, u-PA generally is cleared from the body after less than 1.5 minutes. By using the platelets to deliver only the amount of protein transferred by pre-incubation into the platelets, the amount of the therapeutic protein delivered and thus the therapeutic treatment itself can be more precisely controlled. This is also true in embodiments using cells other than platelets and proteins other than u-PA.

### VI. EXAMPLES OF THE INVENTION

As illustrated in the examples below, the methods of this invention were employed to modify the biological behavior of platelets by causing these hematopoietic lineage cells to secrete a protein of interest at a site of vascular injury. These examples demonstrate one of the many embodiments that may be provided using the methods and compositions of this invention

### Example 1

### Construction and Expression of Tat-u-PA

Construction and expression of a recombinant fusion protein for use in the present invention, TAT-u-PA is illustrated in FIG. 1A and discussed below.

The cDNA for mouse urokinase-type plasminogen activator (u-PA) in mature u-PA form was amplified by reverse transcriptase-polymerase chain reaction (RT-PCR) from murine renal cell total RNA. This mouse u-PA cDNA also included an in-frame sequence encoding a linker sequence RKRRKR (SEQ ID NO: 1) sequence for later cleavage by the enzyme BiP-1, as well as restriction sites for KpnI and SphI, subsequent subcloning into pTAT-HA vector (Brian Meade, University of California at Davis).

Following digestion of both the cDNA insert and pTAT-HA vector with Kpn I/Sph I, the correct clone (SEQ ID NO: 2) was isolated and sequenced to ensure that no sequencing errors had been inadvertently inserted.

The resulting vector is transfected into the bacterial strain E. coli BL21 DE3 pLys S for protein expression after isopropyl-thio-beta-galactosidase induction. The resulting recombinant protein is purified from culture lysate using the incorporated Histidine tag on a nickel column.

Organization of the final recombinant protein is illustrated in FIG. 1B.

### Example 2

### Introduction of Composition into Platelets

Approximately 1 ml of peripheral blood is withdrawn from wild-type, BALB-C mice into a 1/10^{th} volume of 3.8% sodium citrate. The platelets (about 10⁹ cells/ml blood) are separated from the other components of plasma by washing with saline.

Approximately 1 μg of the TAT-u-PA recombinant protein of Example 1 is added to the washed volume of platelets in 0.5-1 ml plasma, and the platelet/TAT-u-PA mixture is allowed to soak at 37° C. for about 30 minutes to allow the TAT-u-PA to penetrate into the platelets through the cell membrane. Following the 30 minute soaking, the platelets are separated from any excess, extracellular TAT-u-PA by re-centrifugation or on a column.

A small percentage of the treated platelets in a buffer/saline are then contacted with fluorescently-labeled antibodies to cell surface antigen, PECAM-1 or anti P-selectin rabbit anti-mouse antibodies, and are examined in a flow cytometer to determine if the platelets are in an activated or non-activated state following incorporation of the TAT-u-PA protein. If activated, P-selectin is expressed on the platelet surface. Untreated, unactivated and phorbol myristyl acetate-activated platelets serve as negative and positive controls, respectively. Non-activated platelets are preferred because they will store the peptide and target to the lesion.

Additionally, the amount of protein that penetrates into the platelets is calculated by immunoblot analysis of total platelet proteins, as previously described, to measure the amount of protein remaining once the platelets are separated from protein remaining extracellularly in the mixture. About 10⁸-10⁹ platelets, separated from the protein and carrying about 1 μg or 100 pg TAT-u-PA protein, are suspended in 0.5-1 ml plasma and infused immediately.

### Example 3

### Administration of Composition into Patients—Carotid Artery Thrombosis Model

To determine the effect of transfusion of the treated platelets on thrombosis, the carotid artery injury thrombosis model is employed. This model has been used successfully to demonstrate a bleeding diathesis in diverse mouse backgrounds. This approach also permits study of the effect of the secretion of urokinase-type plasminogen activator in platelets on thrombus development and stability.

Ferric chloride-induced arterial injury is performed according to published procedures (Fay, W. P et al, 1994 cited above; Fay, W. P. et al, 1999 Blood 93:1825-1830) in 6-8 week old animals. Briefly, the right common carotid artery is exposed by blunt dissection, and a miniature Doppler flow probe (Model 0.5VB, Transonic Systems, Ithaca, N.Y.) is positioned around the artery. A 1×2 mm² strip of Number 1 Whatman filter paper (Fisher Scientific, Pittsburgh, Pa.) soaked in 10% ferric chloride is then applied to the adventitial surface of the artery for 2 min. The field is flushed with saline, and blood flow is continuously monitored for 30 minutes. The time to the initial complete occlusion and the presence or absence of arterial occlusion at 30 min is recorded.

To study the effects of a platelet transfusion, 1.2-1.5×10⁸ of the platelets of Example 2 is dispersed in 300 μl buffer and infused into the left jugular vein of each wildtype mouse of an experimental set immediately before the ferric chloride patch is applied. A first control set of mice receives buffer in place of the platelets. A second control set of mice receive platelets not treated with TAT-u-PA. Platelets are used within 2 hours of treatment as described in Example 2. Total blood counts are measured immediately before and 2 minutes after platelet infusion.

About 30 minutes after infusion, the animals are examined to compare the effect of infusion with the treated platelets of the present invention. Animals receiving the injury and the treated platelets of the present invention are anticipated to exhibit less arterial occlusion in comparison to the mice receiving injury and no platelet transfusion due to the cleaving of TAT-u-PA in the treated platelets and secretion of the u-PA at the site of injury as delivered by the treated platelets of the present invention.

Additionally peripheral blood withdrawn from the animals receiving platelets of the present invention is examined with fluorescently-labeled antibodies to cell surface antigens as described above and are examined in a flow cytometer to determine if the platelets in vivo are in an activated or non-activated state following incorporation of the TAT-u-PA protein and administration. Activated platelets will expose P-selectin and bind to the anti-P-selectin antibody; they will not participate in clotting.

### Example 4

### Administration of Composition into Patients—Pulmonary Microemboli Model

To determine whether the platelets containing TAT-u-PA are effective on the venous side of the circulation and lead to rapid dissolution of pulmonary microemboli, the following animal model is used.

For purposes of a platelet transfusion, 1.2-1.5×10⁸ of the platelets of Example 2 is dispersed in 300 μl of buffer and infused into the left jugular vein of each wildtype mouse. Platelets are used within 2 hours of treatment as described in Example 2. Total blood counts are measured immediately before and 2 minutes after platelet infusion.

¹²⁵I-labeled human microemboli, 1.5-3.5μ³ in size, are prepared as previously described (Bdeir, K. et al, 2000 Blood 96:1820-1826). Wildtype animals are injected with these particles within 48 hours of preparation and within 0.5-6 hours after platelet transfusion.

A first control set of mice receives buffer in place of the platelets. A second control set of mice receives platelet transfusion only with no microemboli injection.

At various time points (2-60 minutes) after microemboli are administered, the animals are euthanized, the lungs removed, washed free of blood, and the amount of ¹²⁵I activity measured using a ZM Coulter Counter™ instrument (Coulter Electronics, Hialeah, Fla.). In other experiments, autoradiograms of lungs are taken from wildtype and transgenic mice 30 minutes after injection of the microemboli by exposing the lungs to X-OMAT™ film (Kodak, Rochester N.Y.).

The results of these examinations are compared to identify any effect of infusion with the treated platelets of the present invention. Animals receiving the microemboli injection and the treated platelets of the present invention are anticipated to exhibit less residual labeled microemboli in comparison to the mice receiving microemboli and no platelet transfusion due to the cleaving of TAT-u-Pa in the treated platelets and secretion of the u-Pa at the site of emboli formation as delivered by the treated platelets of the present invention.

Additionally, peripheral blood withdrawn from the animals receiving platelets of the present invention is examined with fluorescently-labeled antibodies to cell surface antigens as described above and are examined in a flow cytometer to determine if the platelets in vivo are in an activated or non-activated state following incorporation of the TAT-u-PA protein and administration.

### Example 5

### Delivery of Urokinase-Type Plasminogen Activator Decreases Oxygen-Induced Lung Injury in Mice

Inhibition of fibrinolytic activity and intra-alveolar fibrin deposition is important in the development of oxygen (O₂)-induced lung injury. Delivery of urokinase-type plasminogen activator (u-PA) was evaluated in transgenic mice expressing u-PA ectopically in platelet granules in which u-PA is released only when platelets are activated. Such mice (referred to as u-PA⁺ or MUK) are described in Kufrin et al, 2003 Blood 102(3):926-933, incorporated by reference. Since pulmonary sequestration of activated inflammatory cells, including neutrophils (PMN) and platelets, occurs early in response to O₂, and given the ability of the transgenic platelets to enhance fibrin lysis, it was proposed that lung injury would be decreased in the transgenic mice.

The mouse model of hyperoxia involves exposing Control mice (C57/BL6, n=12) to atmospheric oxygen in a sealed plexiglass chamber for up to 180 hours. Wildtype mice (C57/BL6, n-12) and 7-8 wk old female u-PA-expressing mice (“u-PA⁺”; n=12) were exposed to 100% O₂ in a sealed plexiglass chamber. At various times (e.g., times 0, 24 hours, 48 hours, 72 hours, and 96 hours) animals were removed for measurements of lung injury.

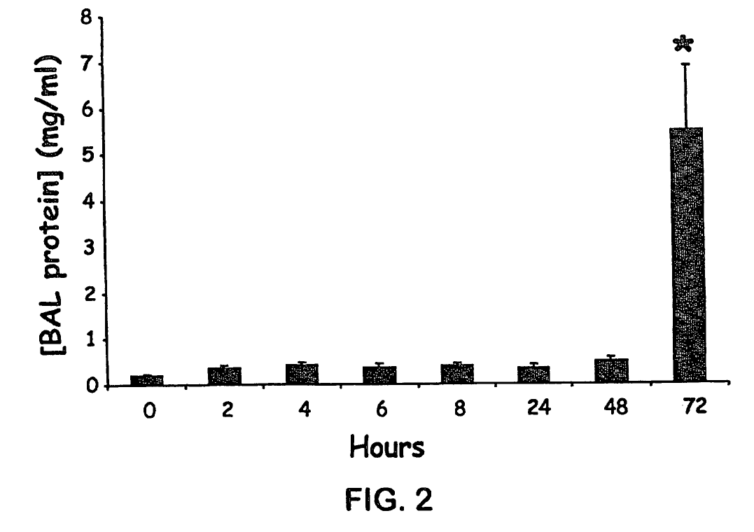

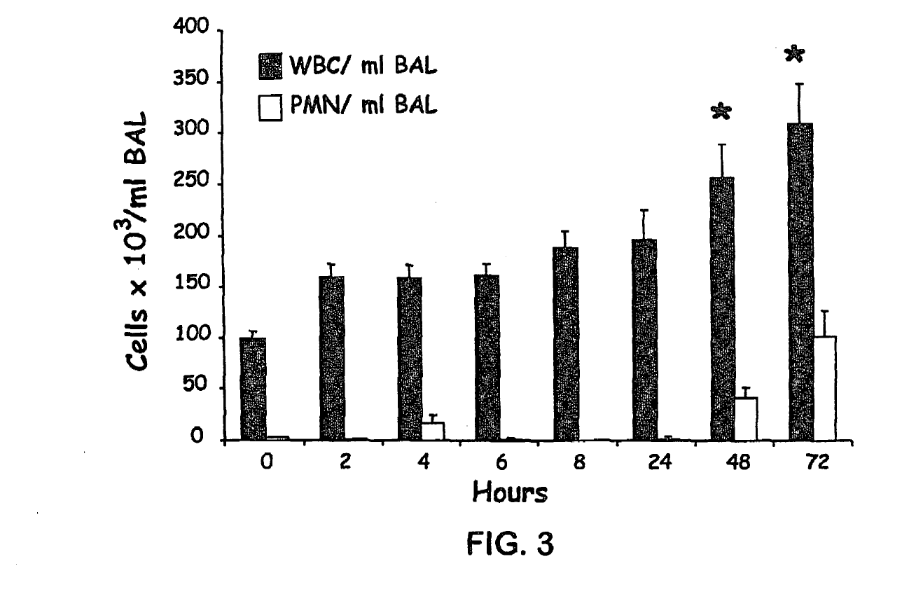

An examination of the lungs indicated increased cellularity evident at 24 hours. By 48 hours in the chamber fibrin deposition is observed in the lung tissue. By 72 hours in the chamber alveolar damage and hemorrhage are observed. FIG. 2 illustrates the BAL protein concentration (mg/ml) in the lungs over time. The asterisk in the figure indicates the p<0.05 vs. baseline. FIG. 3 is a graph indicating leukocyte kinetics in BAL, with the light bars indicating white blood cells/ml BAL and the dark bars indicated polymorphonuclear cells/ml BAL. Cell numbers are indicated at a value×10³/ml BAL. The asterisk in the figure indicates the p<0.05 vs. baseline.

Other data (not shown) is generated by Western blot, and shows that exposure to greater than 95% oxygen increases lung fibrinogen deposition over time and most significantly after 48 hours of exposure of the mice. These results were subsequently confirmed with radiolabeled fibrinogen in the mouse model.

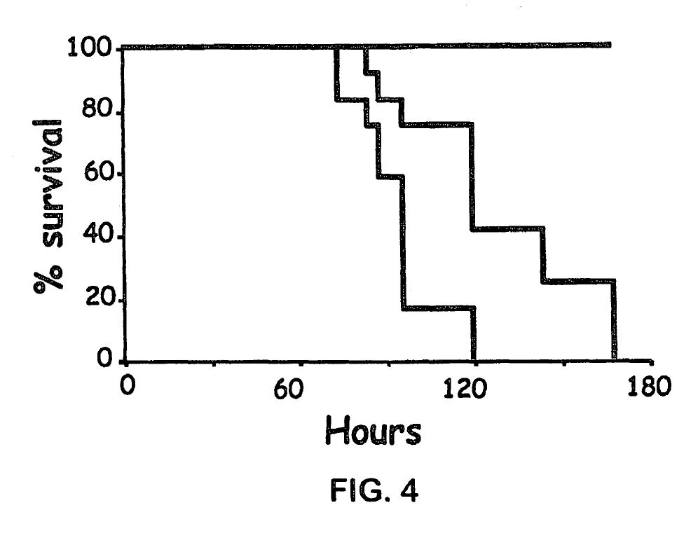

Still other results were depicted in FIG. 4, showing survival times in hyperoxia according to the mouse model described above. The control mice were WT mice exposed to only atmospheric O₂, and are indicated by the horizontal line at 100% survival, indicating that all control mice survived over a 72 hour period. The survival of the WT mice is shown by the stepwise vertical line showing 0% survival at about 120 hours. The stepwise vertical line showing 0% survival at about 170 hours represents the survival of the u-PA⁺ mice. This data demonstrate that provision of additional u-PA extended the survival duration of mice subjected to hyperoxia.

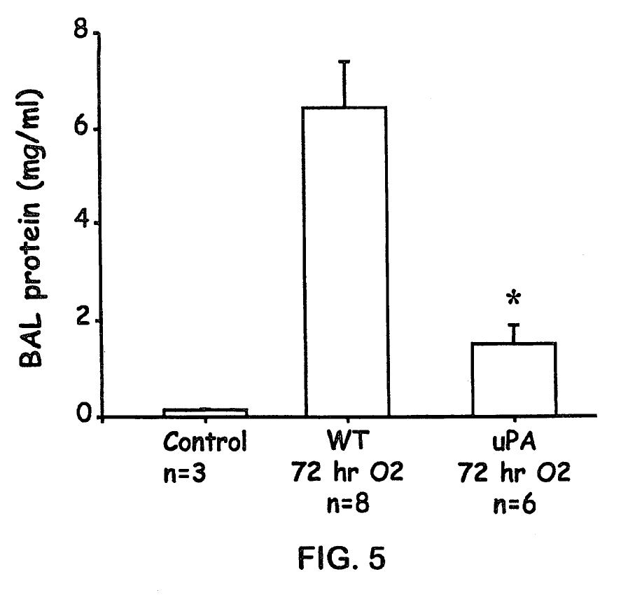

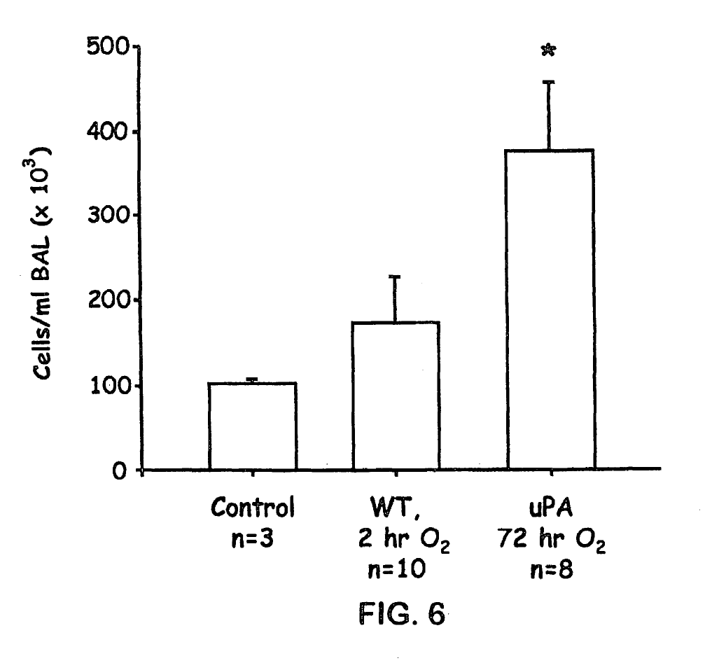

BAL protein concentration for the same groups of mice, namely control mice (n=3), WT mice (n=8) and u-PA⁺ mice (n=6) exposed to 100% O₂ for 72 hours shown in FIG. 5 also demonstrated the benefits of u-PA in mice undergoing lung injury.

BAL WBC counts for the same groups of mice, namely control mice (n=3), WT mice (n=10) and u-PA+ mice (n=8) exposed to 100% O₂ further indicates the benefits of u-PA in treating lung injury.

Data in Table I shown were generated from another experiment using the model model of hyperoxia, specifically generated using 7-8 wk old female u-PA-expressing mice (“u-PA”; n=3) vs. wildtype controls (“WT”; C57/B6, n=5) after 72 hours of exposure to >95% O₂ (mean±SEM).

TABLE 1

| | BAL WBC | BAL PMN | BAL Protein |
|---|---|---|---|
| Mice | Cells/ml(×10³) | (%) | mg/ml |
| u-PA⁺ | 209 ± 71* | 34 ± 9* | 2.2 ± 0.4* |
| WT | 48 ± 8 | 10 ± 3 | 5.7 ± 0.7 |

*denotes a significant difference from WT mice (p < 0.05).

The data appearing in the Table and Figure discussed above demonstrate that lung injury, as assessed by bronchioalveolar lavage fluid (BAL) protein concentration and subjective morphologic lung injury scores, was significantly decreased in u-PA-expressing mice vs. controls. In addition, total WBC counts and percent neutrophils in the BAL were significantly higher, suggesting that increased pulmonary inflammation is not necessarily associated with increased lung injury.

Taken with the knowledge that depletion of circulating PMN and attenuation of PMN influx into the airway does not prevent O₂-induced lung injury, these data provide support that a decrease in lung fibrinolytic activity is a primary factor in the development of O₂-induced lung injury. Although the u-PA was expressed in the cells of the transgenic mice in this experiment, this data serves as proof of principle that lung fibrinolytic activity may be increased by delivery of u-PA. Thus, this experiment demonstrates the desirability of the method of this invention in which non-autologous platelets can be used to deliver, rather than express, u-PA as the therapeutic protein useful in the composition. Compositions of this invention which enable practice of such a method are valuable therapeutic commodities. Using platelets as a delivery system for urokinase plaminogen activator protein should target fibrinolytic activity to the lung and decrease O₂-induced lung injury.

### Example 6

### Stroke Model

Wild type control mice (C57/BL6, n=1) or mUK (mouse platelet-u-PA-expressing mice; n=1) underwent carotid injury and thrombosis using a minimally invasive laser-induced injury model to study thrombus development in mice in vivo. Specifically, the method used was the Rose Bengal/lasar injury method in which low-intensity laser illumination of mice injected with Rose Bengal dye to induce photochemical injury in the region of laser illumination. This method was conducted substantially according to E. D. Rosen et al, 2001, Amer. J. Pathol., 158:1613-1622.

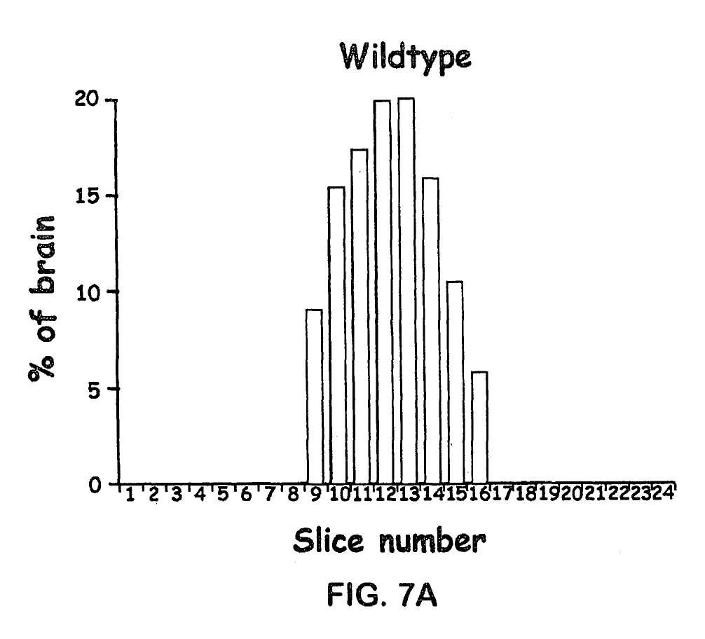

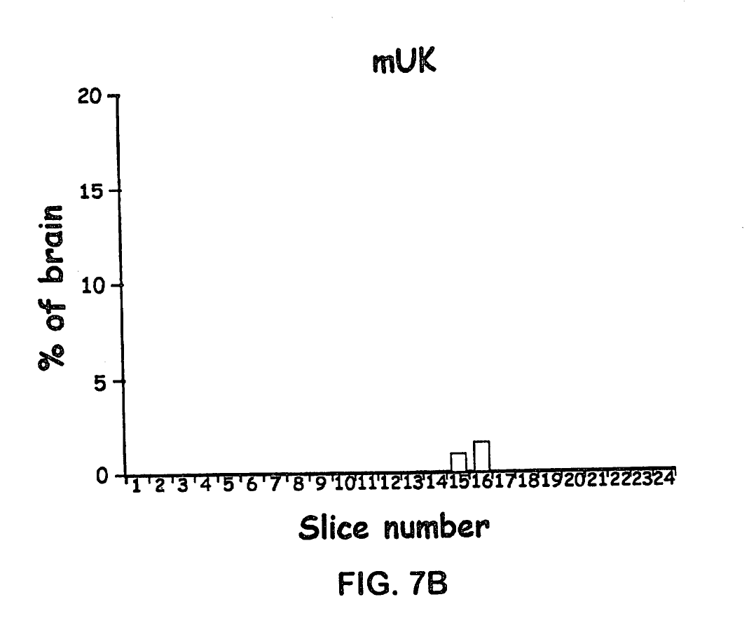

Magnetic resonance images (MRIs; data not shown) were taken at 18 hours post-surgery for both mice. The MRIs of the WT mouse showed a large infarct in the carotid artery. The extent of the infarcted area in serial images for the WT mouse is reported in the bar graph of FIG. 7A, which plots % of brain area affected by the infarct vs. “slice” number. The MRIs of the mUK (u-PA⁺) mouse showed, in comparison, a tiny lesion in the carotid artery. The extent of the infarcted area in serial images for the mUK mice is reported in a corresponding bar graph of FIG. 7B.

This data demonstrate that urokinase plaminogen activator also protects a subject from arterial damage. Thus, using the delivery system of the composition and method of this invention is a useful system to treat photochemical-induced arterial injury.

### Example 7

### Use of Platelets to Transport Protein to Site of Arterial Injury

A. Production of Protein

Drosophila melanogaster S2 cells were transfected with either a cDNA encoding mouse urokinase plasminogen activator (u-PA) (Kufrin et al, 2003 Blood, 102(3):926-33, incorporated by reference herein) or a cDNA encoding Tat-u-PA, as described in Example 1. Stable S2 cell transfectants were selected using the pCoHygro plasmid at 1:20 ratio to cDNA-encoding plasmid followed by selection in complete Drosophila Schneider medium supplemented with 10% fetal bovine serum and 300 μg/ml Hygromycin B. After incubating these stable S2 co-transfectants in Drosophila-SFM media (Invitrogen Corp., Cat. No. 10797-025) supplemented (per liter) with 85 ml of 200 mM L-glutamine (Invitrogen Corp., Cat. No. 25030-081), 10 ml of Penicillin/Streptomycin solution (Invitrogen Corp., Cat. No. 15140-122), and 5 ml of Pluronic F-68 solution (Invitrogen Corp., Cat. No. 24040-032) for 5 days at 22-24° C., sodium dodecyl sulfate polyacrylamide gel electrophoresis (SDS-PAGE) analysis was performed on samples of (a) media from S2 cells transfected with cDNA encoding mouse urokinase (2 μg), (b) media from S2 cells transfected with TAT-u-PA prior to induction with copper (5 μL), or (c) media from S2 cells transfected with TAT-u-PA after induction with copper (5 μL). The gel (data not shown) revealed for lane (a) a 50 kDa band corresponding to full-length uPA. No band is present in lane (b) for the uninduced media. Lane (c) revealed both a 50 kDa blot representative of full-length u-PA and a 30 kDa blot corresponds to the heavy chain of low molecular weight u-PA.

B. Transfer of Protein into Platelets

Samples of platelets retrieved from wild type C57/BL6 mice (containing about 2×10⁷ platelets/mL) in 100 μL were incubated for 30 minutes at 22° C. with media (5 μL) from S2 cells transfected with TAT-u-PA prior to induction with copper or media (5 μL) from S2 cells transfected with TAT-u-PA after induction with copper.

C. Carotid Artery Injury Model

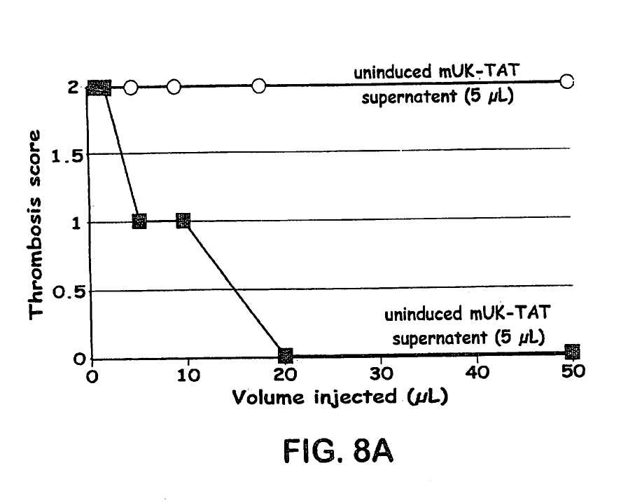

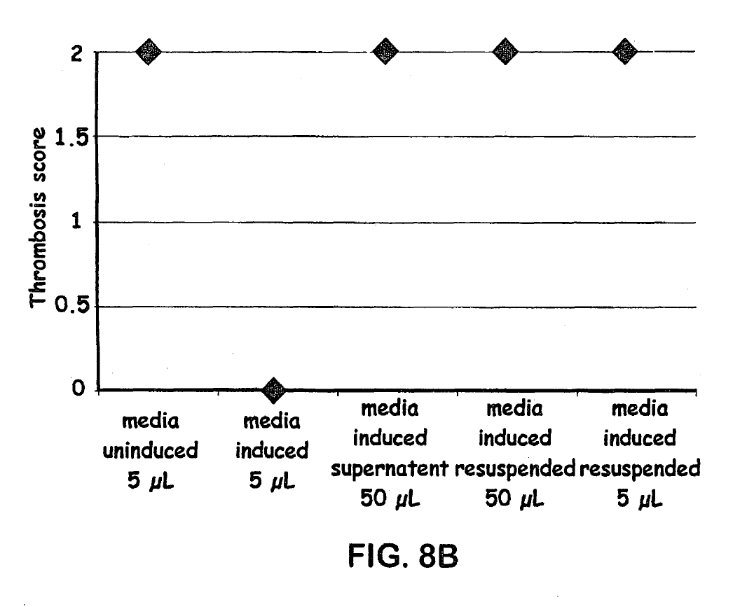

Aliquots of platelets preincubated with uninduced and induced media (O through 50 μL) were injected intravenously into naive WT mice 5 minutes prior to carotid artery injury which was induced by application of ferric chloride, as described in Example 3. Data from one mouse for each of the two conditions, representative of two studies, is shown in FIG. 8A. The thrombosis score is shown on the ordinate as defined below the figure. The thrombosis score extends from 0 through 2, with the score of 0 representing no clot formation over a 30 minute observation; the score of 1 representing an unstable clot formation; and the score of 2 representing stable clot formation. These data show that platelets incubated with pre-induced media containing TAT-u-PA protect against formation of stable occlusive carotid artery thrombosis; whereas platelets pre-incubated with uninduced media (and thereby containing no TAT-u-PA) has no effect upon the formation of stable clots.

### Example 8

### Source of Thrombolytic Activity

This experiment was performed, following the experiment of Example 7, to determine if the thrombolytic activity revealed by the administration of platelets pre-incubated with induced media containing TAT-u-PA was cell associated or was released or was TAT-u-PA not incorporated within platelets.

Platelets were incubated with uninduced and induced media from TAT-u-PA-expressing S2 cells, as described in Example 8. The cells were washed and resuspended in 134 mM NaCl, 3 mM KCl, 0.3 mN NaH₂PO₄, 5 mM HEPES, 5 mM glucose, 2 mM MgCl₂, 7.5% NaHCO₂, bovine serum albumin 300 mg/150 mL final, pH 7.2. The cells were then pelleted at 10000 g for 4 minutes and the pellet solubilized in SDS-PAGE buffer. u-PA associated with platelets before and after pelleting were separated by SDS-PAGE. Plasminogen and milk substrate were added to the gel. The presence of u-PA is denoted by a plasmin-dependent zone of lysis. The gel (data not shown) was run using a size marker in lane 1, uninduced media (5 μL) in lane 2, induced media (5 μL in lane 3), u-PA in platelets before pelleting (5 μL in lane 4) and spun media (5 μL in lane 5). A 50 kDa blot was revealed in lanes 3, 4 and 5, with fainter 30 kDa blots in lanes 3 and 5. No blots were detected in lanes 1 and 2.

Mice were injected with either the supernatant or resuspended cellular pellet composed of platelets that had been preincubated with TAT-u-PA-containing induced media (5 or μL) or uninduced media (containing no TAT-u-PA; 5 μL). Ferric chloride injury was induced as described above in Example 7. As shown by FIG. 8B, only the pellet containing platelets that had been preincubated with TAT-u-PA containing induced media (5 μL) was thrombolytic.

The data from one mouse representative of two such studies is shown in FIG. 8B. These data show that platelets incorporated TAT-u-PA when incubated with induced media. Approximately half of the TAT-u-PA was unincorporated under these conditions (i.e., soluble and is present in the supernatant after platelets are pelleted), and the rest was stably cell-associated (remained in the pellets). The unincorporated-soluble TAT-u-PA that was present in the supernatant or “resuspended” material which had been subjected to vigorous centrifugation, had no effect on the subsequent formation of stable occlusive thrombi, likely because of the rapid (1-2 minute) clearance of u-PA from the blood. In contrast, the pelleted, platelet-associated TAT-u-PA afforded total protection against carotid artery occlusion.

Without wishing to be bound by theory, the inventors believe that the TAT-u-PA found in the supernatant after the vigorous pelleting treatment is believed to be loosely, non-specifically, reversibly associated with cell membrane. The presence of the TAT-u-PA in the supernatant may also be a result of leakage from platelets fractured by the vigorous pelleting conditions. In either instance, the protein present in the supernatant is rapidly cleared and is thus ineffective for thrombolysis, in contrast with the behavior of approximately equal amounts that remained associated with, and likely incorporated within the, platelet. The incorporated protein was released or secreted from the platelet upon activation of the platelet at the site of arterial injury

All publications cited in this specification are incorporated herein by reference. While the invention has been described with reference to a particularly preferred embodiment, it will be appreciated that modifications can be made without departing from the spirit of the invention. Such modifications are intended to fall within the scope of the appended claims.

1. The composition according to claim 12, which comprises a hematopoietic cell or collection of cells differentiated from a common myeloid progenitor cell, said cell or cells containing said non-naturally occurring composition comprising a therapeutic peptide or protein, a transport moiety capable of transporting said first peptide or protein into said cell or cells, and a linker between said first protein and said transport moiety, said linker susceptible to cleavage by an intracellular enzyme in the cell.

2. The composition according to claim 1, wherein said cell or cells are not activated or are activated cell or cells.

3. (canceled)

4. The composition according to claim 1, wherein said cell is a platelet and wherein said therapeutic protein or peptide is selected from the group consisting of Factor VIIa, Factor VIII, Factor IX, fibrinogen, urokinase plasminogen activator, plasminogen, tissue plasminogen activator, and tissue factor pathway inhibitor.

5. (canceled)

6. The composition according to claim 1, wherein said cell is a neutrophil, and wherein said therapeutic protein or peptide is selected from the group consisting of urokinase plasminogen activator receptor and activated Protein C.

7. (canceled)

8. The composition according to claim 1, wherein said cell is an eosinophil and wherein said therapeutic protein or peptide is selected from the group consisting of a protein or fragment thereof toxic to a helminth, human TSG6, an antibody to IL-1 receptor alpha and an anti-inflammatory protein.

9. (canceled)

10. The composition according to claim 1, wherein said cell is an NK cell and wherein said therapeutic protein or peptide is a neutralizing antibody against a viral coat protein.

11. The composition according to claim 12, comprising a pharmaceutically acceptable carrier.

12. A composition comprising a therapeutic peptide or protein, a transport moiety capable of transporting said first peptide or protein into a hematopoietic cell, and an optional linker between said first protein and said transport moiety, said linker susceptible to cleavage by an intracellular enzyme.

13. The composition according to claim 12, wherein said hematopoietic cell is selected from the group consisting a cell differentiated from a common myeloid progenitor cell, a neutrophil, an eosinophil, a basophil, a monocyte, an immature dendritic cell, a mast cell, a macrophage, a dendritic cell, a megakaryocyte, an erythroblast, and a platelet.

14-17. (canceled)

18. The composition according to claim 12, wherein said hematopoietic cell releases the contents of its granules upon activation.

19. The composition according to claim 12, wherein said therapeutic peptide or protein is a fibrinolytic protein selected from the group consisting of urokinase-type plasminogen activator and t-PA.

20. (canceled)

21. The composition according to claim 12, wherein said first peptide or protein is a procoagulant protein selected from the group consisting of Factor VIIa, Factor VIII, Factor IX and fibrinogen.

22. (canceled)

23. The composition according to claim 12, wherein said transport moiety is capable of transporting said therapeutic first peptide or protein into the cell without activating the cell.

24. The composition according to claim 12 wherein said transport moiety is a small, positively charged protein or peptide selected from the group consisting of a transactivating (TAT) protein, a Chariot™ protein, and an arginine-rich peptide.

25-27. (canceled)

28. The composition according to claim 12, wherein said intracellular enzyme is selected from the group consisting of endoplasmic reticulum proteases and serine proteases.

29. The composition according to claim 28 wherein said endoplasmic reticulum protease is BiP.

30. The composition according to claim 28, wherein said serine protease is plasmin.

31. A method for generating a cell or a collection of hematopoietic cells derived from a common myeloid progenitor and capable of delivering a therapeutic protein or peptide to a mammalian patient comprising
transferring a composition of claim 12 into said cell by contacting said cell or a collection of said cells with multiple copies of said composition for sufficient time to permit said compositions to be transported into said cells;
separating said cells from any excess extracellular composition following said contacting step;
adding a pharmaceutically acceptable carrier to said separated cells; and
lyophilizing said cells and carrier.

32-34. (canceled)

35. A method for treating a disorder in a mammalian subject comprising delivering to said subject a composition according to claim 1.

36. The method according to claim 35, wherein said cells are autologous cells or heterologous cells harvested from bone marrow or peripheral blood of said subject.

37. (canceled)

38. The method according to claim 35, wherein said disorder is selected from the group consisting of an infection, inflammation, a vascular injury, acute lung injury, a parasitic helminth infection, asthma, an allergic response, viral infection and any disorders involving or mediated by cells of the hematopoietic lineage.

39. The method according to claim 35, wherein said cell cleaves said first protein or peptide from said composition intracellularly and secretes said first protein or peptide at a suitable site in said subject.

40. The method according to claim 35 comprising reinfusing said cells into the bone marrow or blood of said subject.

41. (canceled)

42. A method for treating or preventing thrombus formation in a mammal comprising delivering to a mammalian patient a platelet or collection of platelets that contain a composition comprising a fibrinolytic peptide or protein, a transport moiety capable of transporting said first peptide or protein into a platelet, and a linker between said first protein and said transport moiety which is susceptible to cleavage by an intracellular enzyme in said platelet, wherein said fibrinolytic protein is selected from the group consisting of urokinase-type plasminogen activator, Factor VIIa, Factor VIII, Factor IX and fibrinogen.

43. (canceled)

44. A method for enhancing coagulation in a mammal, said method comprising delivering to the mammalian patient a hematopoietic cell or collection of cells that contain a composition of claim 12, wherein said cell is a neutrophil and said first peptide/protein is urokinase plasminogen activator receptor.

45. (canceled)

46. A method for preventing or reducing coagulation in a mammalian subject, said method comprising delivering to the mammalian patient a hematopoietic cell or collection of cells that contain a composition of claim 1, wherein said cell is a platelet and said first peptide/protein is urokinase plasminogen activator, plasminogen, tissue plasminogen activator, or tissue factor pathway inhibitor.

47-55. (canceled)

56. A method for the treatment and prevention of undesirable thrombus development in a mammalian patient by administering to the patient a platelet or collection of platelets that contain a composition of claim 1, wherein said composition comprises the recombinant protein TAT-u-PA.

57. (canceled)


SEQUENCE LISTING (2 sequences)

SEQ ID NO: 1 - protein, 6 aa, Artificial
  linker sequence
RKRRKR

SEQ ID NO: 2 - DNA, 2041 bp, Artificial
  plasmid vector containing mouse urokinase-type plasminogen activator
gggatttcctctagaatattttgtttactttaagaaggagatatacatatgcggggttctcatcatcatcatcatcatggtatggctagcatgactggtggacagcaaatgggtcgggatctgtacgacgatgacgataaggatcgatggggatccaagcttggctacggccgcaagaaacgccgccagcgccgccgcggtggatccaccatgtccggctatccatatgacgtcccagactatgctggctccatggccggtacccgtaaacgccgtaaacgcggcagtgtacttggagctcctgatgaatcaaactgtggctgtcagaacggaggtgtatgcgtgtcctacaagtacttctccagaattcgccgatgcagctgcccaaggaaattccagggggagcactgtgagatagatgcatcaaaaacctgctatcatggaaatggtgactcttaccgaggaaaggccaacactgataccaaaggtcggccctgcctggcctggaatgcgcctgctgtccttcagaaaccctacaatgcccacagacctgatgctattagcctaggcctggggaaacacaattactgcaggaaccctgacaaccagaagcgaccctggtgctatgtgcagattggcctaaggcagtttgtccaagaatgcatggtgcatgactgctctcttagcaaaaagccttcttcgtctgtagaccaacaaggcttccagtgtggccagaaggctctaaggccccgctttaagattgttgggggagaattcactgaggtggagaaccagccctggttcgcagccatctaccagaagaacaagggaggaagtcctccctcctttaaatgtggtgggagtctcatcagtccttgctgggtggccagtgccgcacactgcttcattcaactcccaaagaaggaaaactacgttgtctacctgggtcagtcgaaggagagctcctataatcctggagagatgaagtttgaggtggagcagctcatcttgcacgaatactacagggaagacagcctggcctaccataatgatattgccttgctgaagatacgtaccagcacgggccaatgtgcacagccatccaggtccatacagaccatctgcctgcccccaaggtttactgatgctccgtttggttcagactgtgagatcactggctttggaaaagagtctgaaagtgactatctctatccaaagaacctgaaaatgtccgtcgtaaagcttgtttctcatgaacagtgtatgcagccccactactatggctctgaaattaattataaaatgctgtgtgctgcggacccagagtggaaaacagattcctgcaagggcgattctggaggaccgcttatctgtaacatcgaaggccgcccaactctgagtgggattgtgagctggggccgaggatgtgcagagaaaaacaagcccggtgtctacacgagggtctcacacttcctggactggattcaatcccacattggagaagagaaaggtctggccttctgatggccctcaggtagctgagggaagaaacagatgggtcacttgttcccatgcatgcggtgaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctctctttagggttccgatttagtgctttacggcacctcgactccaaaaaacttgattagggtgatggttcacgtagtggg